(19) United States
(12) Patent Application Publication
(10) Pub. No.: US 2023/0321094 A1
(43) Pub. Date: Oct. 12, 2023

(54) COMBINATION THERAPIES WITH OLIG2 INHIBITORS

(71) Applicant: Curtana Pharmaceuticals, Inc., Austin, TX (US)

(72) Inventors: Graham BEATON, Austin, TX (US); Gregory Douglas STEIN, Austin, TX (US); Gordon Robert ALTON, Austin, TX (US)

(21) Appl. No.: 18/042,497

(22) PCT Filed: Aug. 23, 2021
(86) PCT No.: PCT/US2021/047178

§ 371 Date: Feb. 22, 2023

Related U.S. Application Data
(60) Provisional application No. 63/069,472, filed on Aug. 24, 2020.

(51) Int. Cl. A61K 31/505; A61K 45/06; A61P 35/00

(52) U.S. Cl. CPC A61K 31/505; A61K 45/06; A61P 35/00

(57) ABSTRACT

Described herein are pharmaceutical compositions containing compounds which inhibit the activity of Olig2 in combination with a second therapeutic agent. Also described herein are methods of using such pharmaceutical compositions for treating cancer and other diseases.

### CROSS-REFERENCE

This application claims the benefit of U.S. Provisional Application No. 63/069,472, filed on Aug. 24, 2020, which is incorporated herein by reference in its entirety.

### BACKGROUND OF THE INVENTION

Current brain tumor therapeutic agents, which are only able to extend median survival of patients by six months, cause significant systemic toxicity. This toxicity results in serious long term morbidity of the few patients that survive, in terms of cognition, endocrine disorders, and motor effects. Currently brain tumors are essentially incurable with a median survival of fifteen months.

### SUMMARY OF THE INVENTION

In one aspect, described herein is a pharmaceutical composition comprising 1) a first therapeutic agent, 2) a second therapeutic agent, and 3) at least one pharmaceutically acceptable excipient, wherein the first therapeutic agent is a compound of Formula (I), or a pharmaceutically acceptable salt, solvate, or prodrug thereof, having the structure:

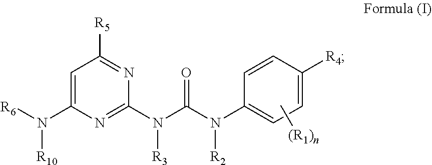

wherein:
   each R₁ is independently halogen, —CN, —NO₂, —OH, —OCF₃, —OCH₂F, —OCF₂H, —SR₈, —N(R₈)S(═O)₂R₉, —S(═O)₂N(R₈)₂, —S(═O)R₉, —S(═O)₂R₉, —C(═O)R₉, —CO₂R₈, —N(R₈)₂, —C(═O)N(R₈)₂, —N(R₈)C(═O)R₉, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl; or two R₁ are taken together to form a substituted or unsubstituted heterocyclic ring or a substituted or unsubstituted carbocyclic ring;
   R₂ and R₃ are each independently H, —CN, C₁-C₄alkyl, C₃-C₆cycloalkyl, or C₂-C₇heterocycloalkyl; or R₂ and R₃ are taken together to form a 5- or 6-membered heterocyclic ring;
   R₄ is H, halogen, —CN, —NO₂, —OH, —OCF₃, —OCH₂F, —OCF₂H, —SR₈, —N(R₈)S(═O)₂R₉, —S(═O)₂N(R₈)₂, —S(═O)R₉, —S(═O)₂R₉, —C(═O)R₉, —CO₂R₈, —N(R₈)₂, —C(═O)N(R₈)₂, —N(R₈)C(═O)R₉, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl;
   R₅ is halogen, —CN, —OH, —CF₃, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl;
   R₆ is —(C(R₁₄)(R₁₅))ₘN(R₁₁)(R₁₂);
   R₁₁ and R₁₂ are each independently H, or substituted or unsubstituted C₁-C₆alkyl; or R₁₁ and R₁₂ are taken together to form a substituted or unsubstituted 5-, 6-, 7-, or 8-membered heterocyclic ring;
   each R₁₄ and R₁₅ are each independently H, or substituted or unsubstituted C₁-C₆alkyl; or
   R₁₄ and R₁₅ are taken together to form a 4-, 5-, 6-membered cycloalkyl ring;
   each R₈ is independently H, or substituted or unsubstituted C₁-C₆alkyl;
   each R₉ is independently substituted or unsubstituted C₁-C₆alkyl;
   R₁₀ is H, or C₁-C₄alkyl;
   m is 2-6; and
   n is 0-4.

In one embodiment, the pharmaceutical composition comprises a compound of Formula (I) wherein R₂ and R₃ are each independently H, —CN, C₁-C₄alkyl, C₃-C₆cycloalkyl, or C₂-C₇heterocycloalkyl. In another embodiment, the pharmaceutical composition comprises a compound of Formula (I) wherein R₂ and R₃ are each H. In another embodiment, the pharmaceutical composition comprises a compound of Formula (I) wherein R₂ and R₃ are taken together to form a 5- or 6-membered heterocyclic ring. In another embodiment, the pharmaceutical composition comprises a compound of Formula (I) wherein R₂ and R₃ are taken together to form a 5-membered heterocyclic ring. In another embodiment, the pharmaceutical composition comprises a compound of Formula (I) wherein R₆ is —(C(R₁₄)(R₁₅))ₘN(R₁₁)(R₁₂) and R₁₄ and R₁₅ are each H. In another embodiment, the pharmaceutical composition comprises a compound of Formula (I) wherein R₁ and R₁₂ are each independently H, or substituted or unsubstituted C₁-C₆alkyl. In another embodiment, the pharmaceutical composition comprises a compound of Formula (I) wherein R₁₁ and R₁₂ are each unsubstituted C₁-C₆alkyl. In another embodiment, the pharmaceutical composition comprises a compound of Formula (I) wherein R₁₁ and R₁₂ are each —CH₃. In another embodiment, the pharmaceutical composition comprises a compound of Formula (I) wherein m is 2. In another embodiment, the pharmaceutical composition comprises a compound of Formula (I) wherein m is 3. In another embodiment, the pharmaceutical composition comprises a compound of Formula (I) wherein R₁₀ is H or CH₃. In another embodiment, the pharmaceutical composition comprises a compound of Formula (I) wherein R₅ is substituted or unsubstituted C₁-C₆alkyl. In another embodiment, the pharmaceutical composition comprises a compound of Formula (I) wherein R₅ is CH₃. In another embodiment, the pharmaceutical composition comprises a compound of Formula (I) wherein R₅ is CH₂CH₃. In another embodiment, the pharmaceutical composition comprises a compound of Formula (I) wherein R₄ is H, halogen, —CN, —NO₂, —OH, —OCF₃, —OCH₂F, —OCF₂H, —SR₈, —N(R₈)S(═O)₂R₉, —S(═O)₂N(R₈)₂, —S(═O)R₉, —S(═O)₂R₉, —C(═O)R₉, —CO₂R₈, —N(R₈)₂, —C(═O)N(R₈)₂, —N(R₈)C(═O)R₉, substituted or unsubstituted C₁-C₆alkyl, or substituted or unsubstituted C₁-C₆alkoxy. In another embodiment, the pharmaceutical composition comprises a compound of Formula (I) wherein R₄ is H, halogen, —CN, —OH, —OCF₃, substituted or unsubstituted C₁-C₆alkyl, or substituted or unsubstituted C₁-C₆alkoxy. In another embodiment, the pharmaceutical composition comprises a compound of Formula (I) wherein R₄ is H, halogen, —OCF₃, substituted or unsubstituted C₁-C₆alkyl, substituted or unsubstituted C₁-C₆alkoxy. In another embodiment, the pharmaceutical composition comprises a compound of Formula (I) wherein R₄ is halogen. In another embodiment, the pharmaceutical composition comprises a compound of Formula (I) wherein each R₁ is independently halogen, —CN, —NO₂, —OH, —OCF₃, —OCH₂F, —OCF₂H, —SR₈, —N(R₈)S(═O)₂R₉, —S(═O)₂N(R₈)₂, —S(═O)R₉, —S(═O)₂R₉, —C(═O)R₉, —CO₂R₈, —N(R₈)₂, —C(═O)N(R₈)₂, —N(R₈)C(═O)R₉, substituted or unsubstituted C₁-C₆alkyl, or substituted or unsubstituted C₁-C₆alkoxy. In another embodiment, the pharmaceutical composition comprises a compound of Formula (I) wherein each R₁ is independently halogen, —CN, —OH, —OCF₃, substituted or unsubstituted C₁-C₆alkyl, or substituted or unsubstituted C₁-C₆alkoxy. In another embodiment, the pharmaceutical composition comprises a compound of Formula (I) wherein each R₁ is independently halogen, —OCF₃, substituted or unsubstituted C₁-C₆alkyl, substituted or unsubstituted C₁-C₆alkoxy. In another embodiment, the pharmaceutical composition comprises a compound of Formula (I) wherein each R₁ is independently is halogen. In another embodiment, the pharmaceutical composition comprises a compound of Formula (I) wherein n is 1. In another embodiment, the pharmaceutical composition comprises a compound of Formula (I) wherein n is 0. In another embodiment, the pharmaceutical composition comprises a compound of Formula (I) wherein n is 0 and R₄ is H, —CN, —NO₂, —OH, —OCF₃, —OCH₂F, —OCF₂H, —SR₈, —N(R₈)S(═O)₂R₉, —S(═O)₂N(R₈)₂, —S(═O)R₉, —S(═O)₂R₉, —C(═O)R₉, —CO₂R₈, —N(R₈)₂, —C(═O)N(R₈)₂, —N(R₈)C(═O)R₉, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl. In another embodiment, the pharmaceutical composition comprises a compound of Formula (I) wherein n is 0 and R₄ is H, —CN, —OH, —OCF₃, substituted or unsubstituted C₁-C₆alkyl, or substituted or unsubstituted C₁-C₆alkoxy. In another embodiment, the pharmaceutical composition comprises a compound of Formula (I) wherein the compound of Formula (I) has the structure:

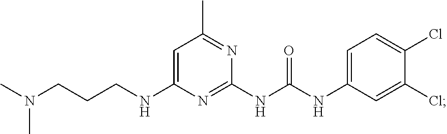 [SMILES: Cc1cc(NCCCN(C)C)nc(NC(=O)Nc2ccc(Cl)c(Cl)c2)n1]

or a pharmaceutically acceptable salt, solvate, or prodrug thereof.

In another aspect, is a pharmaceutical composition comprising 1) a first therapeutic agent, 2) a second therapeutic agent, and 3) at least one pharmaceutically acceptable excipient, wherein the first therapeutic agent has the structure:

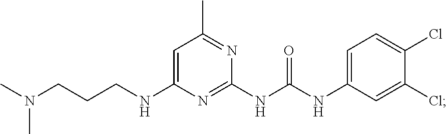 [SMILES: Cc1cc(NCCCN(C)C)nc(NC(=O)Nc2ccc(Cl)c(Cl)c2)n1]

or a pharmaceutically acceptable salt, solvate, or prodrug thereof.

In another embodiment of the aforementioned embodiments, the pharmaceutical composition comprises a second therapeutic agent, wherein the second therapeutic agent is an epidermal growth factor receptor (EGFR) inhibitor, an ataxia telangiectasia mutated (ATM) kinase inhibitor, an ataxia telengiectasia and Rad3 related (ATR) kinase inhibitor, a poly ADP-ribose polymerase (PARP) inhibitor, a cyclin-dependent kinase (CDK) 4/6 inhibitor, a checkpoint kinase 1 (Chk1) inhibitor, a signal transducer and activator of transcription 3 (STAT3) inhibitor, a mechanistic target of rapamycin (mTOR) inhibitor, or a Janus Kinase 2 (JAK2) inhibitor. In another embodiment of the aforementioned embodiments, the pharmaceutical composition comprises a second therapeutic agent, wherein the second therapeutic agent is an epidermal growth factor receptor (EGFR) inhibitor. In another embodiment of the aforementioned embodiments, the pharmaceutical composition comprises a second therapeutic agent, wherein the second therapeutic agent is an ataxia telangiectasia mutated (ATM) kinase inhibitor. In another embodiment of the aforementioned embodiments, the pharmaceutical composition comprises a second therapeutic agent, wherein the second therapeutic agent is an ataxia telengiectasia and Rad3 related (AIR) kinase inhibitor. In another embodiment of the aforementioned embodiments, the pharmaceutical composition comprises a second therapeutic agent, wherein the second therapeutic agent is a poly ADP-ribose polymerase (PARP) inhibitor. In another embodiment of the aforementioned embodiments, the pharmaceutical composition comprises a second therapeutic agent, wherein the second therapeutic agent is a cyclin-dependent kinase (CDK) 4/6 inhibitor. In another embodiment of the aforementioned embodiments, the pharmaceutical composition comprises a second therapeutic agent, wherein the second therapeutic agent is a checkpoint kinase 1 (Chk1) inhibitor. In another embodiment of the aforementioned embodiments, the pharmaceutical composition comprises a second therapeutic agent, wherein the second therapeutic agent is a signal transducer and activator of transcription 3 (STAT3) inhibitor. In another embodiment of the aforementioned embodiments, the pharmaceutical composition comprises a second therapeutic agent, wherein the second therapeutic agent is a mechanistic target of rapamycin (mTOR) inhibitor. In another embodiment of the aforementioned embodiments, the pharmaceutical composition comprises a second therapeutic agent, wherein the second therapeutic agent is a Janus Kinase 2 (JAK2) inhibitor.

In another aspect is a method for treating a disease in a subject comprising administering to the subject in need thereof a pharmaceutical composition described herein, wherein the disease is cancer or Down's Syndrome. In another aspect is a method for treating a disease in a subject comprising administering to the subject in need thereof a pharmaceutical composition comprising 1) a first therapeutic agent, 2) a second therapeutic agent, and 3) at least one pharmaceutically acceptable excipient, wherein the first therapeutic agent is a compound of Formula (I) described herein, or a pharmaceutically acceptable salt, solvate, or prodrug thereof, wherein the disease is cancer or Down's Syndrome. In some embodiments is a method for treating cancer in a subject comprising administering to the subject in need thereof a pharmaceutical composition comprising 1) a first therapeutic agent, 2) a second therapeutic agent, and 3) at least one pharmaceutically acceptable excipient, wherein the first therapeutic agent is a compound of Formula (I) described herein, or a pharmaceutically acceptable salt, solvate, or prodrug thereof. In some embodiments is a method for treating cancer in a subject comprising administering to the subject in need thereof a pharmaceutical composition comprising 1) a first therapeutic agent, 2) a second therapeutic agent, and 3) at least one pharmaceutically acceptable excipient, wherein the first therapeutic agent is a compound of Formula (I) described herein, or a pharmaceutically acceptable salt, solvate, or prodrug thereof, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments is a method for treating Down's Syndrome in a subject comprising administering to the subject in need thereof a pharmaceutical composition comprising 1) a first therapeutic agent, 2) a second therapeutic agent, and 3) at least one pharmaceutically acceptable excipient, wherein the first therapeutic agent is a compound of Formula (I) described herein, or a pharmaceutically acceptable salt, solvate, or prodrug thereof.

In another aspect is a method for treating cancer or Down's Syndrome in a subject comprising administering to the subject in need thereof a pharmaceutical composition comprising 1) a first therapeutic agent, 2) a second therapeutic agent, and 3) at least one pharmaceutically acceptable excipient, wherein the first therapeutic agent is a compound of Formula (I) described herein, or a pharmaceutically acceptable salt, solvate, or prodrug thereof, and the second therapeutic agent is an epidermal growth factor receptor (EGFR) inhibitor, an ataxia telangiectasia mutated (ATM) kinase inhibitor, an ataxia telengiectasia and Rad3 related (ATR) kinase inhibitor, a poly ADP-ribose polymerase (PARP) inhibitor, a cyclin-dependent kinase (CDK) 4/6 inhibitor, a checkpoint kinase 1 (Chk1) inhibitor, a signal transducer and activator of transcription 3 (STAT3) inhibitor, a mechanistic target of rapamycin (mTOR) inhibitor, or a Janus Kinase 2 (JAK2) inhibitor. In some embodiments is a method for treating cancer or Down's Syndrome in a subject comprising administering to the subject in need thereof a pharmaceutical composition comprising 1) a first therapeutic agent, 2) a second therapeutic agent, and 3) at least one pharmaceutically acceptable excipient, wherein the first therapeutic agent is a compound of Formula (I) described herein, or a pharmaceutically acceptable salt, solvate, or prodrug thereof, and the second therapeutic agent is an epidermal growth factor receptor (EGFR) inhibitor. In some embodiments is a method for treating cancer or Down's Syndrome in a subject comprising administering to the subject in need thereof a pharmaceutical composition comprising 1) a first therapeutic agent, 2) a second therapeutic agent, and 3) at least one pharmaceutically acceptable excipient, wherein the first therapeutic agent is a compound of Formula (I) described herein, or a pharmaceutically acceptable salt, solvate, or prodrug thereof, and the second therapeutic agent is an ataxia telangiectasia mutated (ATM) kinase inhibitor. In some embodiments is a method for treating cancer or Down's Syndrome in a subject comprising administering to the subject in need thereof a pharmaceutical composition comprising 1) a first therapeutic agent, 2) a second therapeutic agent, and 3) at least one pharmaceutically acceptable excipient, wherein the first therapeutic agent is a compound of Formula (I) described herein, or a pharmaceutically acceptable salt, solvate, or prodrug thereof, and the second therapeutic agent is an ataxia telengiectasia and Rad3 related (ATR) kinase inhibitor. In some embodiments is a method for treating cancer or Down's Syndrome in a subject comprising administering to the subject in need thereof a pharmaceutical composition comprising 1) a first therapeutic agent, 2) a second therapeutic agent, and 3) at least one pharmaceutically acceptable excipient, wherein the first therapeutic agent is a compound of Formula (I) described herein, or a pharmaceutically acceptable salt, solvate, or prodrug thereof, and the second therapeutic agent is a poly ADP-ribose polymerase (PARP) inhibitor. In some embodiments is a method for treating cancer or Down's Syndrome in a subject comprising administering to the subject in need thereof a pharmaceutical composition comprising 1) a first therapeutic agent, 2) a second therapeutic agent, and 3) at least one pharmaceutically acceptable excipient, wherein the first therapeutic agent is a compound of Formula (I) described herein, or a pharmaceutically acceptable salt, solvate, or prodrug thereof, and the second therapeutic agent is a cyclin-dependent kinase (CDK) 4/6 inhibitor. In some embodiments is a method for treating cancer or Down's Syndrome in a subject comprising administering to the subject in need thereof a pharmaceutical composition comprising 1) a first therapeutic agent, 2) a second therapeutic agent, and 3) at least one pharmaceutically acceptable excipient, wherein the first therapeutic agent is a compound of Formula (I) described herein, or a pharmaceutically acceptable salt, solvate, or prodrug thereof, and the second therapeutic agent is a checkpoint kinase 1 (Chk1) inhibitor. In some embodiments is a method for treating cancer or Down's Syndrome in a subject comprising administering to the subject in need thereof a pharmaceutical composition comprising 1) a first therapeutic agent, 2) a second therapeutic agent, and 3) at least one pharmaceutically acceptable excipient, wherein the first therapeutic agent is a compound of Formula (I) described herein, or a pharmaceutically acceptable salt, solvate, or prodrug thereof, and the second therapeutic agent is a signal transducer and activator of transcription 3 (STAT3) inhibitor. In some embodiments is a method for treating cancer or Down's Syndrome in a subject comprising administering to the subject in need thereof a pharmaceutical composition comprising 1) a first therapeutic agent, 2) a second therapeutic agent, and 3) at least one pharmaceutically acceptable excipient, wherein the first therapeutic agent is a compound of Formula (I) described herein, or a pharmaceutically acceptable salt, solvate, or prodrug thereof, and the second therapeutic agent is a mechanistic target of rapamycin (mTOR) inhibitor. In some embodiments is a method for treating cancer or Down's Syndrome in a subject comprising administering to the subject in need thereof a pharmaceutical composition comprising 1) a first therapeutic agent, 2) a second therapeutic agent, and 3) at least one pharmaceutically acceptable excipient, wherein the first therapeutic agent is a compound of Formula (I) described herein, or a pharmaceutically acceptable salt, solvate, or prodrug thereof, and the second therapeutic agent is a Janus Kinase 2 (JAK2) inhibitor.

In another aspect is the use of a pharmaceutical composition comprising 1) a first therapeutic agent, 2) a second therapeutic agent, and 3) at least one pharmaceutically acceptable excipient, wherein the first therapeutic agent is a compound of Formula (I) described herein, or a pharmaceutically acceptable salt, solvate, or prodrug thereof, in the manufacture of a medicament for the treatment of cancer or Down's Syndrome.

Other objects, features and advantages of the compounds, compositions, methods, and uses described herein will become apparent from the following detailed description. It should be understood, however, that the detailed description and the specific examples, while indicating specific embodiments, are given by way of illustration only, since various changes and modifications within the spirit and scope of the disclosure will become apparent from this detailed description.

### DETAILED DESCRIPTION

Disclosed herein is a pharmaceutical composition comprising 1) a first therapeutic agent, 2) a second therapeutic agent, and 3) at least one pharmaceutically acceptable excipient, wherein the first therapeutic agent is a compound of Formula (I) described herein, or a pharmaceutically acceptable salt, solvate, or prodrug thereof. Further disclosed herein is a pharmaceutical composition comprising 1) a first therapeutic agent that is an OLIG2 modulator having the structure of Formula (I) described herein; 2) a second therapeutic agent that is an epidermal growth factor receptor (EGFR) inhibitor, an ataxia telangiectasia mutated (ATM) kinase inhibitor, an ataxia telengiectasia and Rad3 related (ATR) kinase inhibitor, a poly ADP-ribose polymerase (PARP) inhibitor, a cyclin-dependent kinase (CDK) 4/6 inhibitor, a checkpoint kinase 1 (Chk1) inhibitor, signal transducer and activator of transcription 3 (STAT3) inhibitor, a mechanistic target of rapamycin (mTOR) inhibitor, or a Janus Kinase 2 (JAK2) inhibitor; and 3) at least one pharmaceutically acceptable excipient.

### OLIG2 Biology

The compounds of Formula (I) or (II) described herein are modulators or inhibitors of the neural and GBM (glioblastoma multiforme) stem cell transcriptional repressor OLIG2 (e.g. NM_005806, NP_005797 for human). OLIG2 (also written herein as Olig2) is the oligodendrocyte transcription factor 2. This protein is a member of the bHLH (basic helix-loop-helix) family. The bHLH family is a family of transcription factors that contain the structure motif characterized by two alpha helices connected by a loop. The transcription factors containing bHLH domains are generally dimeric. Generally one of the helices contains basic amino acid residues that facilitate binding to DNA. OLIG2 is normally restricted to the central nervous system (CNS) in non-disease states, where it is an essential regulator of progenitor cell fate. OLIG2 homodimerizes and hetereodimerizes with the E12 or E47 proteins to then bind and repress the p21 gene promoter among other effects. P21 is a stem cell and tumor suppressor, and is directly repressed by OLIG2. P21 is activated by the tumor suppressor p53. p53 occurs in the intact, wild type form in nearly 70% of primary GBM patient samples. OLIG2 is highly expressed in all diffuse gliomas, and is found in virtually 100% of GBM cells positive for the CD133 stem cell marker. Importantly, OLIG2 is typically not found in normal brain and in tissues outside the CNS unless they are malignant, such as T-cell leukemia, melanoma, lung, and breast cancer. No other neural or glial marker gene, and no other transcriptional repressor displays as consistent a link to brain cancers. In contrast, membrane receptors (EGFR, PDGFR, etc) are not uniformly expressed among patients, and various approaches to targeting them has been met with limited success in GBM treatment.

The expression of Olig2 in diffuse gliomas likely results from the transformed stem cell origin of these tumors. It has been found that a small cohort of the cells present in patient GBM expresses neural stem cell markers including CD133 and nestin, among others. The CD133(+) cells isolated from existing GBM are highly tumorigenic when orthotopically implanted into mice. In one study, as few as 100 of the CD133(+) cells extracted from a patient GBM produced an invasive tumor when transplanted into the brain of a recipient mouse, while 100,000 CD133(−) GBM cells were unable to generate a tumor. Consistent with these findings, a strikingly high percentage of GBM occur in close proximity to the neural stem cell germinal zones in the brain, i.e., neural stem cells undergo malignant transformation and migrate some distance from the germinal zones and establish a GBM.

Another significant finding with respect to GBM cancer stem cells (CSCs) is that the CD133(+) cells are significantly more resistant to radiation and cytotoxic agents used to treat GBM than the bulk of the tumor mass which is comprised of CD133(−) cells. This suggests that conventional radio/chemotherapy spares the CSCs within a GBM, and unless these cells are targeted, the tumor invariably is resurgent, with lethal effect. Moreover, the very few patients that survive GBM suffer lifelong morbidity from chemo- and radio-toxicity, in terms of cognition, endocrine balance, and other functions.

Olig2 is highly expressed in GBM CSCs, but is only expressed in low levels by normal brain and is not detected in tissues outside the nervous system. Olig2 inhibitors would offer a therapeutic margin superior to conventional chemotherapy. Low systemic toxicity would be much more compatible with long-term clinical management of GBM than is the case with currently used treatment approaches.

High rates of mortality for patients with brain cancers make this disease a leading cause of cancer related death in men, women, and children. Primary brain tumors are actually the most common solid tumor of childhood and the second leading cause of cancer death after leukemia. The toxicity of current treatments causes serious life-long morbidity in the few patients that survive. The development of small molecule, orally available drugs with low toxicity effective in brain cancers would represent a significant advance. Moreover, the compounds may also be effective in other cancers that are stem cell driven and which highly express Olig2. These cancers include T-cell leukemias, skin cancers, small cell lung cancers, and breast cancers. Moreover, these cancers often metastasize to the brain. This would be relevant to millions of patients worldwide.

In some embodiments described herein, are small molecules that inhibit Olig2 which is a transcription factor critical for survival and proliferation of glioblastoma and other brain cancers, i.e., medulloblastoma, astrocytomas, brain stem gliomas, meningiomas, and oligodendrogliomas. Olig2 especially is detected primarily in the brain, generally not outside the nervous system, and it is highly expressed in glioblastoma tumors. This means that Olig2 inhibition should have relatively low toxicity to a patient. Olig2 is also over-expressed in melanomas, lung cancers, breast cancer, and T-cell leukemias, so an Olig2 inhibitor may also be applicable to the treatment of these cancers.

No other transcription factor or marker displays as consistent a link to brain cancer as does Olig2, so Olig2 inhibition should compare favorably to other signaling pathway inhibitors in glioblastoma. Olig2 is a robust target in that the hinge region of its dimerization loop is unique compared to other proteins of its class (basic helix-loop-helix proteins).

The Olig2 targeted inhibitors described herein should prove unique in terms of efficacy and toxicity.

The existing agents, therapeutics, and methods used to treat brain cancers include Temozolomide (TMZ—Temodar), radiation, cyclophosphamide, carmustine, carboplatin, and occasional supplementation with Avastin. All these are only somewhat effective standard brain cancer therapeutic agents, and they are very toxic. No brain cancer stem cell inhibitors currently exist for brain tumors.

In another aspect, methods of inhibiting the activity of OLIG2 are provided. The methods include contacting an Olig2 protein with an effective amount of a compound provided herein (e.g., a compound of Formula (I) or (II). The compound may have the structure of the Formulae provided herein (or any of the embodiments thereof described above). In some embodiments, the methods of inhibiting an Olig2 protein are conducted within a cell. Thus, in certain embodiments, methods of inhibiting the activity of Olig2 within a cell are provided. The method includes contacting a cell with an effective amount of a compound provided herein. The compound may have the structure of the Formulae provided herein (or any of the embodiments thereof described above). In some embodiments, the cell is a prokaryote or eukaryote. The cell may be a eukaryote (e.g. protozoan cell, fungal cell, plant cell, or an animal cell). In some embodiments, the cell is a mammalian cell such as a human cell, cow cell, pig cell, horse cell, dog cell, cat cell, mouse cell, or rat cell. In some embodiments, the cell is a human cell. The cell may form part of an organ or an organism. In certain embodiments, the cell does not form part of an organ or an organism.

In another aspect, a method of inhibiting the activity of Olig2 in a cell is provided. The method includes contacting the cell with a compound as provided herein (e.g. Formula (I) and (II)). In some embodiments the compound binds the hinge region of the dimerization loop of Olig2. In some embodiments, the compound inhibits dimerization of Olig2.

### EGFR Biology

The human epidermal growth factor receptor (EGFR also known as HER-1 or Erb-B1) is a 170 kDa transmembrane receptor encoded by the c-erbB protooncogene, and exhibits intrinsic tyrosine kinase activity (Modjtahedi et al., Br. J. Cancer 73:228-235 (1996); Herbst and Shin, Cancer 94:1593-1611 (2002)). EGFR regulates numerous cellular processes via tyrosine-kinase mediated signal transduction pathways, including, but not limited to, activation of signal transduction pathways that control cell proliferation, differentiation, cell survival, apoptosis, angiogenesis, mitogenesis, and metastasis (Atalay et al., Ann. Oncology 14:1346-1363 (2003); Tsao and Herbst, Signal 4:4-9 (2003); Herbst and Shin, Cancer 94:1593-1611 (2002); Modjtahedi et al., Br. J. Cancer 73:228-235 (1996)).

Overexpression of EGFR has been reported in numerous human malignant conditions, including cancers of the bladder, brain, head and neck, pancreas, lung, breast, ovary, colon, prostate, and kidney. (Atalay et al., Ann. Oncology 14:1346-1363 (2003); Herbst and Shin, Cancer 94:1593-1611 (2002); and Modjtahedi et al., Br. J. Cancer 73:228-235 (1996)). In many of these conditions, the overexpression of EGFR correlates or is associated with poor prognosis of the patients. EGFR is also expressed in the cells of normal tissues, particularly the epithelial tissues of the skin, liver, and gastrointestinal tract, although at generally lower levels than in malignant cells (See Herbst and Shin).

### ATM Biology

Ataxia telangiectasia mutated (ATM) is a serine/threonine protein kinase that is recruited and activated by DNA double-strand breaks (DSBs). It phosphorylates several key proteins that initiate activation of the DNA damage checkpoint, leading to cell cycle arrest, DNA repair or apoptosis. Several of these targets, including p53, CHK2, BRCA1, NBS1, and H2AX, are tumor suppressors. The ATM protein is a 350 kDa polypeptide that is a member of the phosphatidylinositol (PI) 3-kinase family of proteins by virtue of a putative kinase domain in its carboxyl-terminal region. ATM is the product of the gene mutated in ataxia-telangiectasia (AT). Ataxia telangiectasia (AT) is a rare human disease characterized by cerebellar degeneration, extreme cellular sensitivity to radiation, and a predisposition to cancer. All AT patients contain mutations in the ATM gene (ATM). Most other AT-like disorders are defective in genes encoding the MRN protein complex. One feature of the ATM protein is its rapid increase in kinase activity immediately following double-strand break formation. The phenotypic manifestation of AT is due to the broad range of substrates for the ATM kinase, involving DNA repair, apoptosis, G1/S, intra-S checkpoint and G2/M checkpoints, gene regulation, translation initiation, and telomere maintenance. Therefore, a defect in ATM has severe consequences in repairing certain types of damage to DNA, and cancer may result from improper repair. AT patients have an increased risk for breast cancer that has been ascribed to ATM's interaction and phosphorylation of BRCA1 and its associated proteins following DNA damage. Certain kinds of leukemias and lymphomas, including Mantle cell lymphoma, T-ALL, atypical B cell chronic lymphocytic leukemia, and T-PLL are also associated with ATM defects.

### ATR Biology

ATR (“ATM and Rad3 related”) kinase is a protein kinase involved in cellular responses to DNA damage. ATR kinase acts with ATM (“ataxia telangiectasia mutated”) kinase and many other proteins to regulate a cell's response to DNA damage, commonly referred to as the DNA Damage Response (“DDR”). The DDR stimulates DNA repair, promotes survival, and stalls cell cycle progression by activating cell cycle checkpoints, which provide time for repair. Without the DDR, cells are much more sensitive to DNA damage and readily die from DNA lesions induced by endogenous cellular processes such as DNA replication or exogenous DNA damaging agents commonly used in cancer therapy. Healthy cells can rely on a host of different proteins for DNA repair including the DDR kinase ATR. In some cases these proteins can compensate for one another by activating functionally redundant DNA repair processes. On the contrary, many cancer cells harbor defects in some of their DNA repair processes, such as ATM signaling, and therefore display a greater reliance on their remaining intact DNA repair proteins which include ATR. In addition, many cancer cells express activated oncogenes or lack key tumor suppressors, and this can make these cancer cells prone to dysregulated phases of DNA replication which in turn cause DNA damage. ATR has been implicated as a critical component of the DDR in response to disrupted DNA replication. As a result, these cancer cells are more dependent on ATR activity for survival than healthy cells. Accordingly, ATR inhibitors may be useful for cancer treatment, because they shut down a DNA repair mechanism that is more important for cellular survival in many cancer cells than in healthy normal cells. Disruption of ATR function (e.g. by gene deletion) has been shown to promote cancer cell death both in the absence and presence of DNA damaging agents. This suggests that ATR inhibitors may be effective both as single agents and as potent sensitizers to radiotherapy or genotoxic chemotherapy.

### PARP Biology

Poly (ADP-ribose) polymerase (PARP) (also referred to as poly(adenosine 5′-diphospho-ribose) polymerase or PARS (poly(ADP-ribose) synthetase) is a family of proteins involved in a number of cellular processes involving mainly DNA repair and programmed cell death. PARP is composed of four domains of interest: a DNA-binding domain, a caspase-cleaved domain, an auto-modification domain, and a catalytic domain. The DNA-binding domain is composed of two zinc finger motifs. Members of the PARP enzyme family comprise PARP-1, PARP-2, PARP-3, and Vault-PARP; and Tankyrases (TANKs), such as, for example: TANK-1 and TANK-2. PARP has an essential role in facilitating DNA repair, controlling RNA transcription, mediating cell death, and regulating immune response. These actions make PARP inhibitors targets for a broad spectrum of disorders. PARP inhibitors have demonstrated efficacy in numerous models of disease, particularly in models of ischemia reperfusion injury, inflammatory disease, degenerative diseases, protection from adverse effects of cytotoxic compounds, and the potentiation of cytotoxic cancer therapy. PARP has also been indicated in retroviral infection and thus inhibitors may have use in antiretroviral therapy. PARP inhibitors have been efficacious in preventing ischemia reperfusion injury in models of myocardial infarction, stroke, other neural trauma, organ transplantation, as well as reperfusion of the eye, kidney, gut, and skeletal muscle. Inhibitors have been efficacious in inflammatory diseases such as arthritis, gout, inflammatory bowel disease, CNS inflammation such as MS and allergic encephalitis, sepsis, septic shock, hemorrhagic shock, pulmonary fibrosis, and uveitis. PARP inhibitors have also shown benefit in several models of degenerative disease including diabetes (as well as complications) and Parkinson's disease. PARP inhibitors can ameliorate the liver toxicity following acetaminophen overdose, cardiac and kidney toxicities from doxorubicin and platinum based antineoplastic agents, as well as skin damage secondary to sulfur mustards. In various cancer models, PARP inhibitors have been shown to potentiate radiation and chemotherapy by increasing apoptosis of cancer cells, limiting tumor growth, decreasing metastasis, and prolonging the survival of tumor-bearing animals.

### CDK Biology

Cyclin-dependent kinases (CDKs) are a family of serine/threonine protein kinases playing important cellular functions. The cyclins are the regulatory subunits that activate the catalytic CDKs. CDK1/Cyclin B1, CDK2/Cyclin A, CDK2/Cyclin E, CDK4/Cyclin D, and CDK6/Cyclin D are critical regulators of cell cycle progression. CDKs also regulate transcription, DNA repair, differentiation, senescence, and apoptosis (Morgan, D. O., Annu. Rev. Cell. Dev. Biol., 13:261-291 (1997)). Small molecule inhibitors of CDKs have been developed to treat cancer (de Carcer, G. et al., Curr. Med. Chem., 14:969-85 (2007)). A large amount of genetic evidence supports that CDKs, their substrates or regulators have been shown to be associated with many human cancers (Malumbres, M. et al, Nature Rev. Cancer, 1:222-231 (2001)). Endogenous protein inhibitors of CDKs including p16, p21, and p27 inhibit CDK activity and their overexpression results in cell cycle arrest and inhibition of tumor growth in preclinical models (Kamb, A., Curr. Top. Microbiolo. Immunol., 227:139-148 (1998)). Small molecule inhibitors of CDKs may also be used to treat variety of other diseases that result from aberrant cell proliferation, including cardiovascular disorders, renal diseases, certain infectious diseases and autoimmune diseases. Cell proliferation pathways including genes involved in the cell cycle G1 and S phase checkpoint (p53, pRb, p15, p16, and Cyclins A, D, E, CDK 2, and CDK4) have been associated with plaque progression, stenosis and restenosis after angioplasty. Over-expression of the CDK inhibitor protein p21 has been shown to inhibit vascular smooth muscle proliferation and intimal hyperplasia following angioplasty (Chang, M. W. et al., J. Clin. Invest., 96:2260 (1995); Yang, Z-Y. et al., Proc. Natl. Acad. Sci. (USA) 93:9905 (1996)). Selective inhibitors of some CDKs may also be used to protect normal untransformed cells by inhibiting specific phases of cell cycle progression (Chen, et al., J. Natl. Cancer Institute, 92:1999-2008 (2000)). Pre-treatment with a selective CDK inhibitor prior to the use of a cytotoxic agent that inhibits a different phase of the cell cycle may reduce the side effects associated with the cytotoxic chemotherapy and possibly increase the therapeutic window. It has been shown that induction of cellular protein inhibitors of CDKs (p16, p27 and p21) conferred strong resistance to paclitaxel- or cisplatin-mediated cytotoxicity on the inhibitor-responsive cells but not on the inhibitor-unresponsive cells (Schmidt, M, Oncogene, 2001 20:6164-71). CDK4 and CDK6 are two functionally indistinguishable cyclin D dependent kinases. They are widely expressed with high levels of expression observed in cells of hematopoietic lineage. CDK4/6 promotes G1-S transition of the cell cycle by phosphorylating the retinoblastoma protein (Rb). CDK4 and CDK6 single knockout mice are viable and double knockout mice die around birth with defective hematopoiesis (Satyanarayana, A. et al., Oncogene, 28:2925-39 (2009); Malumbres, M. et al., Cell, 118:493-504 (2004)). Strong evidence supports a significant involvement of the cyclin D-CDK4-p16INK4A-Rb pathway in cancer development (Malumbres, M. et al., Nature Rev. Cancer, 1:222-31 (2001)). Rb negatively regulates the cell cycle at G1 by sequestering E2F proteins that are required for initiation of S phase. p16INK4A is a key member of the INK4 family of CDK4/6 cellular inhibitors. The genes for Rb and p16INK4A are tumor suppressors that are often deleted or silenced in cancer cells. Additionally CDK4, CDK6, and cyclin D are reported to be amplified in hematologic malignancies and solid tumors. The importance of this pathway in oncogenesis is further supported by the finding that depletion or inactivation of CDK4 inhibits tumor growth in mouse tumor models (Yu, Q. et al., Cancer Cell, 9:23-32 (2006); Puyol, M. Cancer Cell, 18:63-73 (2010)).

### Chk1 Biology

Cell cycle checkpoints are regulatory pathways that control the order and timing of cell cycle transitions that lies downstream from ATM and/or ATR in the DNA damage checkpoint signal transduction pathway. They ensure that critical events such as DNA replication and chromosome segregation are completed in high fidelity. The regulation of these cell cycle checkpoints is a critical determinant of the manner in which tumor cells respond to many chemotherapies and radiation. Many effective cancer therapies work by causing DNA damage; however, resistance to these agents remains a significant limitation in the treatment of cancer. Of the several mechanisms of drug resistance, an important one is attributed to the prevention of cell cycle progression through the control of critical activation of a checkpoint pathway. This arrests the cell cycle to provide time for repair, and induces the transcription of genes to facilitate repair, thereby avoiding immediate cell death. By abrogating checkpoint arrests at, for example, the G2 checkpoint, it may be possible to synergistically augment tumor cell death induced by DNA damage and circumvent resistance. Human Chk-1 plays a role in regulating cell cycle arrest by phosphorylating the phosphatase cdc25 on Serine 216, which may be involved in preventing activation of cdc2/cyclin B and initiating mitosis. Therefore, inhibition of Chk-1 should enhance DNA damaging agents by initiating mitosis before DNA repair is complete and thereby causing tumor cell death.

### JAK Biology

Janus kinases (JAKs) are protein tyrosine kinases ubiquitously expressed in cells. JAKs are involved in membrane signaling events which are triggered by a variety of extracellular factors that interact with cell surface receptors. JAKs initiate the cytoplasmic signal transduction cascades of cytokine receptors that lack a protein tyrosine kinase domain. The signal transduction cascades are initiated after oligomerization of surface receptors due to ligand binding. Cytoplasmic receptor-associated JAKs are then activated which subsequently phosphorylate tyrosine residues along the receptor chains. These phosphotyrosine residues are targets for a variety of SH2 domain-containing transducer proteins, such as the signal transducers and activators of transcription (STAT) proteins. After STAT binds to receptor chains, they are phosphorylated by the JAK proteins, dimerize and translocate into the nucleus. In the nucleus, STAT alter the expression of cytokine-regulated genes.

Mammalian JAK-2 belongs to the JAK kinase family that also includes JAK-1, JAK-3 and TYK-2. JAK-1, JAK-2, and TYK-2 are ubiquitously expressed, while JAK-3 is predominantly expressed in hematopoietic cells. These kinases consist of approximately 1150 amino acids, with molecular weights of about 120 kDa to 130 kDa. The amino acid sequences of the JAK kinase family are characterized by the presence of highly conserved domains. These domains include the JAK homology (JH) domains, C-terminal domain (JH1) responsible for the tyrosine kinase function, the tyrosine kinase-like domain (JH2) that shows high similarity to functional kinases but does not possess any catalytic activity, and the N-terminal domain that spans JH7 to JH3) that is important for receptor association and non-catalytic activity. Although the function of the N-terminal domain is not well established, there is some evidence for a regulatory role on the JH1 domain, thus modulating catalytic activity.

JAK-2 is activated in a wide variety of human cancers such as prostrate, colon, ovarian, breast cancers, melanoma, leukemia and other hematopoietic malignancies. In addition, somatic point mutation of the JAK-2 gene has been identified to be highly associated with classic myeloproliferative disorders (MPD) and in other myeloid disorders. Constitutive activation of JAK-2 activity is also caused by chromosomal translocation in hematopoietic malignancies, such as in TEL-JAK-2 which is primarily associated with T-ALL, and in PCM1-JAK-2 which is associated with B-ALL and CML. It has been shown that inhibition of the JAK/STAT pathway, and in particular inhibition of JAK-2 activity, results in anti-proliferative and pro-apoptotic effects largely due to inhibition of phosphorylation of STAT. Furthermore, inhibition of JAK-2 activity by pharmacological agents or by expression of dominant negative JAK-2 effectively block tumor growth and induce apoptosis by reducing the STAT phosphorylation in cell culture and human tumor xenografts in vivo.

mTOR Biology

Mechanistic target of rapamycin (mTOR) is a kinase within the family of phosphatidylinositol-3 kinase-related kinases (PIKKs), which is a family of serine/threonine protein kinases, with a sequence similarity to the family of lipid kinases, PI3Ks. mTOR is a downstream effector of the PI3K/AKT pathway, and forms two distinct multiprotein complexes, mTORC1 and mTORC2. These two complexes have a separate network of protein partners, feedback loops, substrates, and regulators. mTORC1 consists of mTOR and two positive regulatory subunits, raptor and mammalian LST8 (mLST8), and two negative regulators, proline-rich AKT substrate 40 (PRAS40) and DEPTOR. mTORC2 consists of mTOR, mLST8, mSin1, protor, rictor, and DEPTOR.

Many human tumors occur because of dysregulation of mTOR signaling, and can confer higher susceptibility to inhibitors of mTOR. Deregulations of multiple elements of the mTOR pathway, like PI3K amplification/mutation, PTEN loss of function, AKT overexpression, and S6K1, 4EBP1, and eTF4E overexpression have been related to many types of cancers.

### STAT Biology

Signal transducer and activator of transcription (STAT) proteins were originally discovered as a family of latent cytoplasmic transcription factors that mediate normal cellular responses to cytokines, growth factors, and other polypeptide ligands. The activation of STATs is an important event for the mediation of cytokine and growth factor-induced cellular and biological processes, including proliferation, differentiation, survival, development, and inflammation. Seven members of the STAT family of proteins have been identified in mammalians: STAT1, 2, 3, 4, 5a, 5b, and 6. All of the family members share six distinct structural domains, including the N-terminal, coiled-coil, DNA-binding, Src homology 2 (SH2), and the transactivation domains, and contain a critical tyrosine (Tyr) residue at the C-terminus (Tyr705 for STAT3), which is phosphorylated during activation. STAT activation by phosphorylation is mediated by growth factor receptor tyrosine kinases, and the cytoplasmic kinases, such as cytokine receptor-associated Janus kinases (JAKs), and Src family kinases. Phosphorylation induces STAT:STAT dimer formation between two monomers via a reciprocal phosphoTyr (pTyr)-SH2 domain interactions, although pre-existing complexes between inactive STAT monomers have also been detected. From the cytoplasm, STATs accumulate in the nucleus where they mediate gene transcription by binding to specific DNA response elements.

In contrast to the transient nature of STAT activation in normal cells, many human solid and hematological tumors harbor STAT3 activity and evidence strongly implicates aberrantly active STAT3 in tumor formation. A large body of evidence supports the dysregulation of growth and survival, the promotion of angiogenesis, and the suppression of host's immune surveillance of tumor (Turkson J. Expert Opin Ther Targets. 2004; 8(5):409-22). Moreover, aberrant STAT3 promotes invasion and metastasis, thereby contributing to tumor progression (See Turkson). It is now established that persistently active STAT3 functions as a master regulator of molecular and biological events that promote tumorigenesis. Thus, the inhibition of aberrant STAT3 activity by genetic or pharmacological approaches induced growth arrest and apoptosis of tumor cells in vitro, as well as tumor regression in vivo.

### Certain Terminology

Unless defined otherwise, all technical and scientific terms used herein have the same meaning as is commonly understood to which the claimed subject matter belongs. In the event that there are a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described.

Definition of standard chemistry terms may be found in reference works including, but not limited to, Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology were employed.

Unless specific definitions are provided, the nomenclature employed in connection with, and the laboratory procedures and techniques of, analytical chemistry, synthetic organic chemistry, and medicinal and pharmaceutical chemistry described herein are those recognized in the field. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, compositions described herein.

As used herein, C₁-Cₓ includes C₁-C₂, C₁-C₃ . . . C₁-Cₓ. C₁-Cₓ refers to the number of carbon atoms that make up the moiety to which it designates (excluding optional substituents).

An “alkyl” group refers to an aliphatic hydrocarbon group. The alkyl groups may or may not include units of unsaturation. The alkyl moiety may be a “saturated alkyl” group, which means that it does not contain any units of unsaturation (i.e. a carbon-carbon double bond or a carbon-carbon triple bond). The alkyl group may also be an “unsaturated alkyl” moiety, which means that it contains at least one unit of unsaturation. The alkyl moiety, whether saturated or unsaturated, may be branched, straight chain, or cyclic.

The “alkyl” group may have 1 to 12 carbon atoms (whenever it appears herein, a numerical range such as “1 to 6” refers to each integer in the given range; e.g., “1 to 6 carbon atoms” means that the alkyl group may consist of 1 carbon atom, 2 carbon atoms, 3 carbon atoms, etc., up to and including 6 carbon atoms, although the present definition also covers the occurrence of the term “alkyl” where no numerical range is designated). The alkyl group of the compounds described herein may be designated as “C₁-C₆ alkyl” or similar designations. By way of example only, “C₁-C₆ alkyl” indicates that there are one to six carbon atoms in the alkyl chain, i.e., the alkyl chain is selected from the group consisting of methyl, ethyl, n-propyl, iso-propyl, n-butyl, iso-butyl, sec-butyl, t-butyl, n-pentyl, iso-pentyl, neo-pentyl, hexyl, propen-3-yl (allyl), cyclopropylmethyl, cyclobutylmethyl, cyclopentylmethyl, cyclohexylmethyl. Alkyl groups can be substituted or unsubstituted. Depending on the structure, an alkyl group can be a monoradical or a diradical (i.e., an alkylene group).

An “alkoxy” refers to a “—O-alkyl” group, where alkyl is as defined herein.

The term “alkenyl” refers to a type of alkyl group in which the first two atoms of the alkyl group form a double bond that is not part of an aromatic group. That is, an alkenyl group begins with the atoms —C(R)═CR₂, wherein R refers to the remaining portions of the alkenyl group, which may be the same or different. Non-limiting examples of an alkenyl group include —CH═CH₂, —C(CH₃)═CH₂, —CH═CHCH₃, —CH═C(CH₃)₂ and —C(CH₃)═CHCH₃. The alkenyl moiety may be branched, straight chain, or cyclic (in which case, it would also be known as a “cycloalkenyl” group). Alkenyl groups may have 2 to 6 carbons. Alkenyl groups can be substituted or unsubstituted. Depending on the structure, an alkenyl group can be a monoradical or a diradical (i.e., an alkenylene group).

The term “alkynyl” refers to a type of alkyl group in which the first two atoms of the alkyl group form a triple bond. That is, an alkynyl group begins with the atoms —C≡C—R, wherein R refers to the remaining portions of the alkynyl group. Non-limiting examples of an alkynyl group include —C≡CH, —C≡CCH₃, —C≡CCH₂CH₃ and —C≡CCH₂CH₂CH₃. The “R” portion of the alkynyl moiety may be branched, straight chain, or cyclic. An alkynyl group can have 2 to 6 carbons. Alkynyl groups can be substituted or unsubstituted. Depending on the structure, an alkynyl group can be a monoradical or a diradical (i.e., an alkynylene group).

“Amino” refers to a —NH₂ group.

The term “alkylamine” or “alkylamino” refers to the —N(alkyl)ₓHy group, where alkyl is as defined herein and x and y are selected from the group x=1, y=1 and x=2, y=0. When x=2, the alkyl groups, taken together with the nitrogen to which they are attached, can optionally form a cyclic ring system. “Dialkylamino” refers to a —N(alkyl)₂ group, where alkyl is as defined herein.

The term “aromatic” refers to a planar ring having a delocalized π-electron system containing 4n+2π electrons, where n is an integer. Aromatic rings can be formed from five, six, seven, eight, nine, or more than nine atoms. Aromatics can be optionally substituted. The term “aromatic” includes both aryl groups (e.g., phenyl, naphthalenyl) and heteroaryl groups (e.g., pyridinyl, quinolinyl).

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, and naphthalenyl. Depending on the structure, an aryl group can be a monoradical or a diradical (i.e., an arylene group).

The term “carbocyclic ring” refers to a ring wherein each of the atoms forming the ring is a carbon atom. The carbocyclic ring may be aryl or cycloalkyl.

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to,

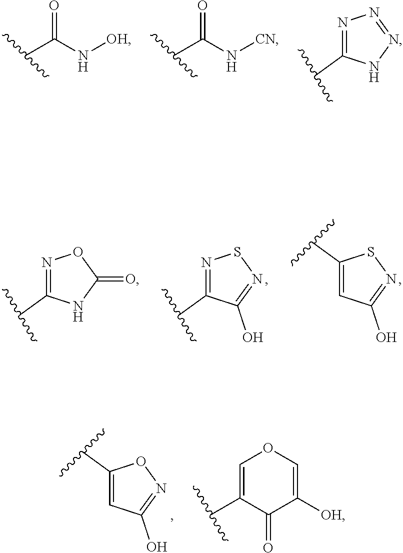

and the like.

The term “cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyls may be fused with an aromatic ring (in which case the cycloalkyl is bonded through a non-aromatic ring carbon atom). Cycloalkyl groups include groups having from 3 to 10 ring atoms. Illustrative examples of cycloalkyl groups include, but are not limited to, the following moieties:

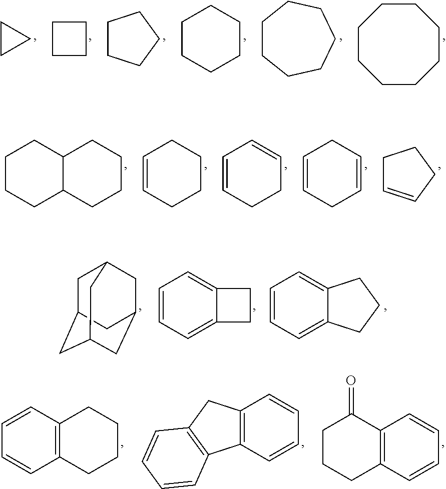

and the like.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. Polycyclic heteroaryl groups may be fused or non-fused. Illustrative examples of heteroaryl groups include the following moieties:

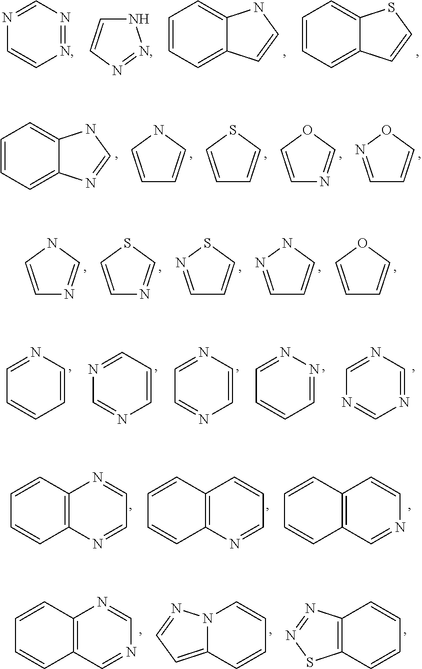

and the like.

A “heterocycloalkyl” group or “heteroalicyclic” group refers to a cycloalkyl group, wherein at least one skeletal ring atom is a heteroatom selected from nitrogen, oxygen and sulfur. The radicals may be fused with an aryl or heteroaryl. Illustrative examples of heterocycloalkyl groups, also referred to as non-aromatic heterocycles, include:

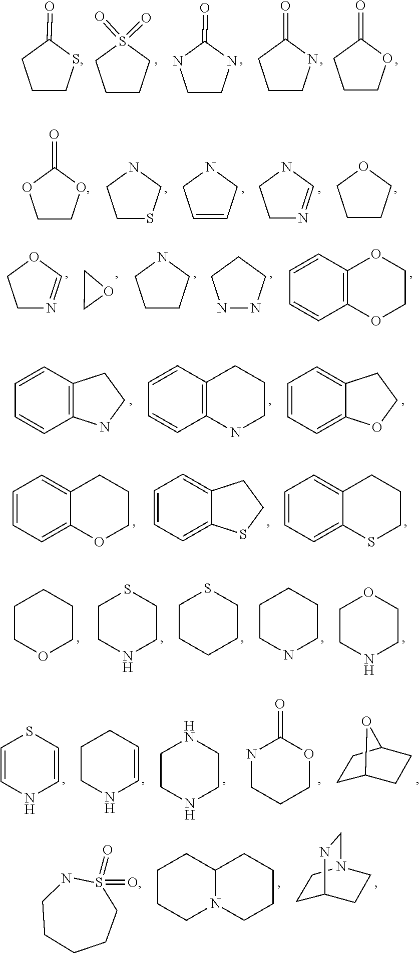

and the like. The term heteroalicyclic also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring).

The term “heterocyclic ring” refers to a ring wherein at least one skeletal ring atom is a heteroatom selected from nitrogen, oxygen and sulfur. The heterocyclic ring may be heteroaryl or heterocycloalkyl.

The term “halo” or, alternatively, “halogen” means fluoro, chloro, bromo and iodo.

The term “haloalkyl” refers to an alkyl group that is substituted with one or more halogens. The halogens may the same or they may be different. Non-limiting examples of haloalkyls include —CH₂Cl, —CF₃, —CHF₂, —CH₂CF₃, —CF₂CF₃, —CF(CH₃)₃, and the like.

The terms “fluoroalkyl” and “fluoroalkoxy” include alkyl and alkoxy groups, respectively, that are substituted with one or more fluorine atoms. Non-limiting examples of fluoroalkyls include —CF₃, —CHF₂, —CH₂F, —CH₂CF₃, —CF₂CF₃, —CF₂CF₂CF₃, —CF(CH₃)₃, and the like. Non-limiting examples of fluoroalkoxy groups, include —OCF₃, —OCHF₂, —OCH₂F, —OCH₂CF₃, —OCF₂CF₃, —OCF₂CF₂CF₃, —OCF(CH₃)₂, and the like.

The term “heteroalkyl” refers to an alkyl radical where one or more skeletal chain atoms is selected from an atom other than carbon, e.g., oxygen, nitrogen, sulfur, phosphorus, silicon, or combinations thereof. The heteroatom(s) may be placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH₂—NH—OCH₃, —CH₂—O—Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃. Excluding the number of heteroatoms, a “heteroalkyl” may have from 1 to 6 carbon atoms.

The term “bond” or “single bond” refers to a chemical bond between two atoms, or two moieties when the atoms joined by the bond are considered to be part of larger substructure.

The term “moiety” refers to a specific segment or functional group of a molecule. Chemical moieties are often recognized chemical entities embedded in or appended to a molecule.

As used herein, the substituent “R” appearing by itself and without a number designation refers to a substituent selected from among from alkyl, haloalkyl, heteroalkyl, alkenyl, cycloalkyl, aryl, heteroaryl (bonded through a ring carbon), and heterocycloalkyl.

The term “optionally substituted” or “substituted” means that the referenced group may be substituted with one or more additional group(s) individually and independently selected from alkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halo, acyl, acyloxy, —CO₂H, —CO₂-alkyl, nitro, haloalkyl, fluoroalkyl, and amino, including mono- and di-substituted amino groups (e.g. —NH₂, —NHR, —N(R)₂), and the protected derivatives thereof. By way of example, an optional substituents may be L^{s}R^{s}, wherein each L^{s} is independently selected from a bond, —O—, —C(═O)—, —S—, —S(═O)—, —S(═O)₂—, —NH—, —NHC(O)—, —C(O)NH—, —S(═O)₂NH—, —NHS(═O)₂—, —OC(O)NH—, —NHC(O)O—, —(C₁-C₆alkyl)-, or —(C₂-C₆alkenyl)-; and each R^{s} is independently selected from among H, (C₁-C₆alkyl), (C₃-C₈cycloalkyl), aryl, heteroaryl, heterocycloalkyl, and C₁-C₆heteroalkyl. The protecting groups that may form the protective derivatives of the above substituents are found in sources such as Greene and Wuts, above.

The methods and formulations described herein include the use of crystalline forms (also known as polymorphs), or pharmaceutically acceptable salts of compounds having the structure of Formula (I) or (II), as well as active metabolites of these compounds having the same type of activity. In some situations, compounds may exist as tautomers. All tautomers are included within the scope of the compounds presented herein. In addition, the compounds described herein can exist in unsolvated as well as solvated forms with pharmaceutically acceptable solvents such as water, ethanol, and the like. The solvated forms of the compounds presented herein are also considered to be disclosed herein.

The terms “kit” and “article of manufacture” are used as synonyms.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “disease” or “condition” refer to a state of being or health status of a patient or subject capable of being treated with the compounds or methods provided herein. In embodiments, the disease is a disease related to (e.g. caused by) Olig2 or aberrant Olig2 activity (e.g. brain cancer, glioblastoma multiforme, medulloblastoma, astrocytomas, brain stem gliomas, meningiomas, oligodendrogliomas, melanomas, lung cancers, breast cancer, leukemias, or Down's Syndrome). Examples of diseases, disorders, or conditions include, but are not limited to brain cancer, glioblastoma multiforme, medulloblastoma, astrocytomas, brain stem gliomas, meningiomas, oligodendrogliomas, melanomas, lung cancers, breast cancer, leukemias, Down's Syndrome, colorectal cancer, papillary thyroid cancer, hepatocellular carcinoma, Alzheimer's disease, Parkinson's disease, Huntington's Disease, frontotemporal dementia, Creutzfeldt-Jakob disease, Gerstmann-Straussler-Scheinker syndrome, prion disease, neurodegenerative diseases, cancer, cardiovascular disease, hypertension, Syndrome X, depression, anxiety, glaucoma, human immunodeficiency virus (HIV) or acquired immunodeficiency syndrome (AIDS), neurodegeneration, Alzheimer's disease, Parkinson's disease, cognition enhancement, Cushing's Syndrome, Addison's Disease, osteoporosis, frailty, muscle frailty, inflammatory diseases, osteoarthritis, rheumatoid arthritis, asthma and rhinitis, adrenal function-related ailments, viral infection, immunodeficiency, immunomodulation, autoimmune diseases, allergies, wound healing, compulsive behavior, multi-drug resistance, addiction, psychosis, anorexia, cachexia, post-traumatic stress syndrome, post-surgical bone fracture, medical catabolism, major psychotic depression, mild cognitive impairment, psychosis, dementia, hyperglycemia, stress disorders, antipsychotic induced weight gain, delirium, cognitive impairment in depressed patients, cognitive deterioration in individuals with Down's syndrome, psychosis associated with interferon-alpha therapy, chronic pain, pain associated with gastroesophageal reflux disease, postpartum psychosis, postpartum depression, neurological disorders in premature infants, migraine headaches, stroke, aneurysm, brain aneurysm, cerebral aneurysm, brain attack, cerebrovascular accident, ischemia, thrombosis, arterial embolism, hemorrhage, transient ischemic attack, anemia, embolism, systemic hypoperfusion, venous thrombosis, arthritis, reperfusion injury, skin diseases or conditions, acne, acne vulgaris, keratosis pilaris, acute, promyelocytic leukemia, baldness, acne rosacea, harlequin ichthyosis, xeroderma pigmentosum, keratoses, neuroblastoma, fibrodysplasia ossificans progressive, eczema, rosacea, sun damage, wrinkles, or cosmetic conditions. In some instances, “disease” or “condition” refer to cancer. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm, or malignant tumors found in mammals, including leukemia, carcinomas, and sarcomas. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus, or Medulloblastoma. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epidermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

A “cancer associated with aberrant Olig2 activity” (also referred to herein as “Olig2 related cancer”) is a cancer caused by aberrant Olig2 activity (e.g. a mutated Olig2 gene). Olig2 related cancers may include brain cancer, glioblastoma multiforme, medulloblastoma, astrocytomas, brain stem gliomas, meningiomas, oligodendrogliomas, melanomas, lung cancers, breast cancer, leukemias, or T cell leukemias.

The terms “treat,” “treating” or “treatment,” as used herein, include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically. For example, in certain methods presented herein successfully treat cancer by decreasing the incidence of cancer and or causing remission of cancer. In some embodiments, certain methods presented herein successfully treat Down's Syndrome by decreasing the incidence of Down's Syndrome or reducing one or more symptoms of Down's Syndrome or reducing the severity of one or more symptoms of Down's Syndrome.

As used herein, amelioration of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

The term “modulate,” as used herein, means to interact with a target protein either directly or indirectly so as to alter the activity of the target protein, including, by way of example only, to inhibit the activity of the target, or to limit or reduce the activity of the target.

As used herein, the term “modulator” refers to a compound that alters an activity of a target. For example, a modulator can cause an increase or decrease in the magnitude of a certain activity of a target compared to the magnitude of the activity in the absence of the modulator. In certain embodiments, a modulator is an inhibitor, which decreases the magnitude of one or more activities of a target. In certain embodiments, an inhibitor completely prevents one or more activities of a target. In some embodiments, an Olig2 modulator is a compound that reduces the activity of Olig2 in a cell. In some embodiments, an Olig2 disease modulator is a compound that reduces the severity of one or more symptoms of a disease associated with Olig2 (e.g. cancer or Down's Syndrome).

As used herein, the term “target activity” refers to a biological activity capable of being modulated by a modulator. Certain exemplary target activities include, but are not limited to, binding affinity, signal transduction, enzymatic activity, tumor growth, inflammation or inflammation-related processes, and amelioration of one or more symptoms associated with a disease or condition.

The terms “inhibits”, “inhibiting”, or “inhibitor” of Olig2 activity, as used herein, refer to inhibition of oligodendrocyte transcription factor 2 activity. In reference to a protein-inhibitor interaction the terms mean negatively affecting (e.g. decreasing) the activity or function of the protein (e.g. decreasing gene transcription regulated by Olig2) relative to the activity or function of the protein (e.g. Olig2, transcription factor) in the absence of the inhibitor (e.g. Olig2 inhibitor or Olig2 inhibitor compound). In some embodiments inhibition refers to reduction of a disease or symptoms of disease. In some embodiments, inhibition refers to a reduction in the activity of a signal transduction pathway or signaling pathway (e.g. reduction of a pathway involving transcription regulation by Olig2 or transcription regulated by Olig2). Thus, inhibition includes, at least in part, partially or totally blocking stimulation, decreasing, preventing, or delaying activation, or inactivating, desensitizing, or down-regulating signal transduction or enzymatic activity or the amount of a protein (e.g. Olig2). In some embodiments, inhibition refers to inhibition of Olig2.

The term “acceptable” with respect to a formulation, composition or ingredient, as used herein, means having no persistent detrimental effect on the general health of the subject being treated.

By “pharmaceutically acceptable,” as used herein, refers a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutical combination” as used herein, means a product that results from the mixing or combining of more than one active ingredient and includes both fixed and non-fixed combinations of the active ingredients. The term “fixed combination” means that one active ingredient, e.g. a compound of Formula (I) or (II), and a co-agent, are both administered to a patient simultaneously in the form of a single entity or dosage. The term “non-fixed combination” means that one active ingredient, e.g. a compound of Formula (I) or (II), and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutical composition” refers to a mixture of a compound of Formula (I) or (II) described herein with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary, and topical administration.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition that includes a compound of Formula (I) or (II) described herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated, however, the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme (e.g. Olig2). In some embodiments, the protein may be Olig2. In some embodiments contacting includes allowing a compound described herein to interact with a protein or enzyme that is involved in transcription.

The terms “co-administration” or the like, as used herein, are meant to encompass administration of the selected therapeutic agents to a single patient, and are intended to include treatment regimens in which the agents are administered by the same or different route of administration or at the same or different time.

The term “excipient” or “carrier,” as used herein, refers to relatively nontoxic chemical compounds or agents that facilitate the incorporation of a compound into cells or tissues.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to, a phosphate buffered saline solution.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines, and free sulphydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds.

“Bioavailability” refers to the percentage of the weight of the compound disclosed herein (e.g. compound of Formula (I) or (II)), that is delivered into the general circulation of the animal or human being studied. The total exposure (AUC(0-∞)) of a drug when administered intravenously is usually defined as 100% bioavailable (F %). “Oral bioavailability” refers to the extent to which a compound disclosed herein, is absorbed into the general circulation when the pharmaceutical composition is taken orally as compared to intravenous injection.

“Blood plasma concentration” refers to the concentration of a compound of Formula (I) or (II) disclosed herein, in the plasma component of blood of a subject. It is understood that the plasma concentration of compounds described herein may vary significantly between subjects, due to variability with respect to metabolism and/or possible interactions with other therapeutic agents. In accordance with one embodiment disclosed herein, the blood plasma concentration of the compounds disclosed herein may vary from subject to subject. Likewise, values such as maximum plasma concentration (Cmax) or time to reach maximum plasma concentration (Tmax), or total area under the plasma concentration time curve (AUC(0-∞)) may vary from subject to subject. Due to this variability, the amount necessary to constitute “a therapeutically effective amount” of a compound may vary from subject to subject.

As used herein, “amelioration” refers to an improvement in a disease or condition or at least a partial relief of symptoms associated with a disease or condition.

As used herein, “immune cells” include cells of the immune system and cells that perform a function or activity in an immune response, such as, but not limited to, T-cells, B-cells, lymphocytes, macrophages, dendritic cells, neutrophils, eosinophils, basophils, mast cells, plasma cells, white blood cells, antigen presenting cells, and natural killer cells.

### Compounds

### Olig2 Modulators:

Described herein are Olig2 modulator compounds having the structure of Formula (I) or (II). In some embodiments described herein is a pharmaceutical composition comprising 1) a first therapeutic agent, 2) a second therapeutic agent, and 3) at least one pharmaceutically acceptable excipient, wherein the first therapeutic agent is a compound of Formula (I), or a pharmaceutically acceptable salt, solvate, or prodrug thereof, having the structure:

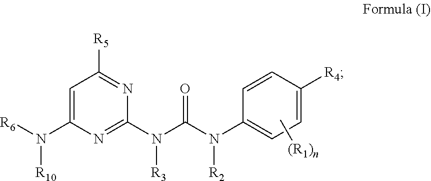

wherein:
   each R₁ is independently halogen, —CN, —NO₂, —OH, —OCF₃, —OCH₂F, —OCF₂H, —SR₈, —N(R₈)S(═O)₂R₉, —S(═O)₂N(R₈)₂, —S(═O)R₉, —S(═O)₂R₉, —C(═O)R₉, —CO₂R₈, —N(R₈)₂, —C(═O)N(R₈)₂, —N(R₈)C(═O)R₉, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl; or two R₁ are taken together to form a substituted or unsubstituted heterocyclic ring or a substituted or unsubstituted carbocyclic ring;
   R₂ and R₃ are each independently H, —CN, C₁-C₄alkyl, C₃-C₆cycloalkyl, or C₂-C₇heterocycloalkyl; or R₂ and R₃ are taken together to form a 5- or 6-membered heterocyclic ring;
   R₄ is H, halogen, —CN, —NO₂, —OH, —OCF₃, —OCH₂F, —OCF₂H, —SR₈, —N(R₈)S(═O)₂R₉, —S(═O)₂N(R₈)₂, —S(═O)R₉, —S(═O)₂R₉, —C(═O)R₉, —CO₂R₈, —N(R₈)₂, —C(═O)N(R₈)₂, —N(R₈)C(═O)R₉, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl;
   R₅ is halogen, —CN, —OH, —CF₃, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl;
   R₆ is —(C(R₁₄)(R₁₅))ₘN(R₁₁)(R₁₂);
   R₁₁ and R₁₂ are each independently H, or substituted or unsubstituted C₁-C₆alkyl; or R₁₁ and R₁₂ are taken together to form a substituted or unsubstituted 5-, 6-, 7-, or 8-membered heterocyclic ring;
   each R₁₄ and R₁₅ are each independently H, or substituted or unsubstituted C₁-C₆alkyl; or
   R₁₄ and R₁₅ are taken together to form a 4-, 5-, 6-membered cycloalkyl ring;
   each R₈ is independently H, or substituted or unsubstituted C₁-C₆alkyl;
   each R₉ is independently substituted or unsubstituted C₁-C₆alkyl;
   R₁₀ is H, or C₁-C₄alkyl;
   m is 2-6; and
   n is 0-4.

In another embodiment is a compound of Formula (I) wherein R₂ and R₃ are each independently H, —CN, C₁-C₄alkyl, C₃-C₆cycloalkyl, or C₂-C₇heterocycloalkyl. In another embodiment is a compound of Formula (I) wherein R₂ and R₃ are each H.

In another embodiment is a compound of Formula (I) wherein R₂ and R₃ are each independently H, —CN, C₁-C₄alkyl, C₃-C₆cycloalkyl, or C₂-C₇heterocycloalkyl; and at least one of R₂ and R₃ is not H. In another embodiment is a compound of Formula (I) wherein R₂ is H, and R₃ is C₁-C₄alkyl. In another embodiment is a compound of Formula (I) wherein R₂ is H, and R₃ is CH₃. In another embodiment is a compound of Formula (I) wherein R₂ is H, and R₃ is C₃-C₆cycloalkyl. In another embodiment is a compound of Formula (I) wherein R₂ is H, and R₃ is cyclopropyl. In another embodiment is a compound of Formula (I) wherein R₂ is H, and R₃ is cyclopentyl. In another embodiment is a compound of Formula (I) wherein R₂ is CH₃, and R₃ is CH₃. In another embodiment is a compound of Formula (I) wherein R₂ is C₁-C₄alkyl, and R₃ is H. In another embodiment is a compound of Formula (I) wherein R₂ is CH₃, and R₃ is H. In another embodiment is a compound of Formula (I) wherein R₂ is C₃-C₆cycloalkyl, and R₃ is H. In another embodiment is a compound of Formula (I) wherein R₂ is cyclopropyl, and R₃ is H. In another embodiment is a compound of Formula (I) wherein R₂ is cyclopentyl, and R₃ is H.

In another embodiment is a compound of Formula (I) wherein R₂ and R₃ are taken together to form a 5- or 6-membered heterocyclic ring. In another embodiment is a compound of Formula (I) wherein R₂ and R₃ are taken together to form a 5-membered heterocyclic ring. In another embodiment is a compound of Formula (I) wherein R₂ and R₃ are taken together to form 6-membered heterocyclic ring.

In another embodiment is a compound of Formula (I) wherein n is 0.

In another embodiment is a compound of Formula (I) wherein each R₁ is independently halogen, —CN, —NO₂, —OH, —OCF₃, —OCH₂F, —OCF₂H, —CF₃, —SR₈, —N(R₈)S(═O)₂R₉, —S(═O)₂N(R₈)₂, —S(═O)R₉, —S(═O)₂R₉, —C(═O)R₉, —CO₂R₈, —N(R₈)₂, —C(═O)N(R₈)₂, —N(R₈)C(═O)R₉, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl. In another embodiment is a compound of Formula (I) wherein each R₁ is independently halogen, —CN, —OH, —CF₃, substituted or unsubstituted C₁-C₆alkyl, or substituted or unsubstituted C₁-C₆alkoxy. In another embodiment is a compound of Formula (I) wherein each R₁ is independently halogen, —CN, —OCF₃, —OCH₂F, —OCF₂H, —CF₃, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl. In another embodiment is a compound of Formula (I) wherein each R₁ is independently halogen, —CN, —OCF₃, —OCH₂F, —OCF₂H, —CF₃, substituted or unsubstituted C₁-C₆alkyl, or substituted or unsubstituted C₁-C₆alkoxy. In another embodiment is a compound of Formula (I) wherein each R₁ is independently halogen, —CN, —OCF₃, —OCH₂F, —OCF₂H, —CF₃, substituted or unsubstituted C₁-C₆alkyl, or substituted or unsubstituted C₁-C₆alkoxy, and n is 3. In another embodiment is a compound of Formula (I) wherein each R₁ is independently halogen, —CN, —OCF₃, —OCH₂F, —OCF₂H, —CF₃, substituted or unsubstituted C₁-C₆alkyl, or substituted or unsubstituted C₁-C₆alkoxy, and n is 2. In another embodiment is a compound of Formula (I) wherein n is 3, and each R₁ is independently halogen. In another embodiment is a compound of Formula (I) wherein n is 2, and each R₁ is independently halogen. In another embodiment is a compound of Formula (I) wherein n is 2, and each R₁ is independently F or Cl. In another embodiment is a compound of Formula (I) wherein n is 2, and each R₁ is F. In another embodiment is a compound of Formula (I) wherein n is 2, and each R₁ is independently Cl. In another embodiment is a compound of Formula (I) wherein n is 2, and each R₁ is independently halogen or —CF₃. In another embodiment is a compound of Formula (I) wherein n is 2, and each R₁ is independently F or —CF₃. In another embodiment is a compound of Formula (I) wherein n is 2, and each R₁ is independently Cl or —CF₃. In another embodiment is a compound of Formula (I) wherein n is 1, and R₁ is halogen. In another embodiment is a compound of Formula (I) wherein n is 1, and R₁ is F. In another embodiment is a compound of Formula (I) wherein n is 1, and R₁ is Cl. In another embodiment is a compound of Formula (I) wherein n is 1, and R₁ is —CF₃. In another embodiment is a compound of Formula (I) wherein n is 1, and R₁ is substituted or unsubstituted C₁-C₆alkyl. In another embodiment is a compound of Formula (I) wherein n is 1, and R₁ is CH₃. In another embodiment is a compound of Formula (I) wherein n is 1, and R₁ is substituted or unsubstituted C₁-C₆alkoxy. In another embodiment is a compound of Formula (I) wherein n is 1, and R₁ is —OCH₃. In another embodiment is a compound of Formula (I) wherein n is 1, and R₁ is —OCF₃. In another embodiment is a compound of Formula (I) wherein n is 1, and R₁ is —OCF₂H.

In another embodiment is a compound of Formula (I) wherein each R₄ is independently halogen, —CN, —NO₂, —OH, —OCF₃, —OCH₂F, —OCF₂H, —CF₃, —SR₈, —N(R₈)S(═O)₂R₉, —S(═O)₂N(R₈)₂, —S(═O)R₉, —S(═O)₂R₉, —C(═O)R₉, —CO₂R₈, —N(R₈)₂, —C(═O)N(R₈)₂, —N(R₈)C(═O)R₉, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl. In another embodiment is a compound of Formula (I) wherein each R₁ is independently halogen, —CN, —OH, —CF₃, substituted or unsubstituted C₁-C₆alkyl, or substituted or unsubstituted C₁-C₆alkoxy. In another embodiment is a compound of Formula (I) wherein each R₁ is independently halogen, —CN, —OCF₃, —OCH₂F, —OCF₂H, —CF₃, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl. In another embodiment is a compound of Formula (I) wherein each R₁ is independently halogen, —CN, —OCF₃, —OCH₂F, —OCF₂H, —CF₃, substituted or unsubstituted C₁-C₆alkyl, or substituted or unsubstituted C₁-C₆alkoxy.

In another embodiment is a compound of Formula (I) wherein R₄ is H, halogen, —CN, —NO₂, —OH, —OCF₃, —OCH₂F, —OCF₂H, —SR₈, —N(R₈)S(═O)₂R₉, —S(═O)₂N(R₈)₂, —S(═O)R₉, —S(═O)₂R₉, —C(═O)R₉, —CO₂R₈, —N(R₈)₂, —C(═O)N(R₈)₂, —N(R₈)C(═O)R₉, substituted or unsubstituted C₁-C₆alkyl, or substituted or unsubstituted C₁-C₆alkoxy. In another embodiment is a compound of Formula (I) wherein R₄ is H, halogen, —CN, —OH, —OCF₃, substituted or unsubstituted C₁-C₆alkyl, or substituted or unsubstituted C₁-C₆alkoxy. In another embodiment is a compound of Formula (I) wherein R₄ is H. In another embodiment is a compound of Formula (I) wherein R₄ is halogen. In another embodiment is a compound of Formula (I) wherein R₄ is F. In another embodiment is a compound of Formula (I) wherein R₄ is Cl. In another embodiment is a compound of Formula (I) wherein R₄ is Br. In another embodiment is a compound of Formula (I) wherein R₄ is —CF₃. In another embodiment is a compound of Formula (I) wherein R₄ is —OCF₃. In another embodiment is a compound of Formula (I) R₄ is —CH₃. In another embodiment is a compound of Formula (I) wherein R₄ is —OCH₃.

In another embodiment is a compound of Formula (I) wherein n is 0 and R₄ is H. In another embodiment is a compound of Formula (I) wherein n is 0 and R₄ is halogen. In another embodiment is a compound of Formula (I) wherein n is 0 and R₄ is F. In another embodiment is a compound of Formula (I) wherein n is 0 and R₄ is Cl. In another embodiment is a compound of Formula (I) wherein n is 0 and R₄ is Br. In another embodiment is a compound of Formula (I) wherein n is 0 and R₄ is —CF₃. In another embodiment is a compound of Formula (I) wherein n is 0 and R₄ is —OCF₃. In another embodiment is a compound of Formula (I) wherein n is 0 and R₄ is —CH₃. In another embodiment is a compound of Formula (I) wherein n is 0 and R₄ is —OCH₃.

In another embodiment is a compound of Formula (I) wherein when n is 0, then R₄ is not halogen. In another embodiment is a compound of Formula (I) wherein n is 0 and R₄ is H, —CN, —NO₂, —OH, —OCF₃, —OCH₂F, —OCF₂H, —SR₈, —N(R₈)S(═O)₂R₉, —S(═O)₂N(R₈)₂, —S(═O)R₉, —S(═O)₂R₉, —C(═O)R₉, —CO₂R₈, —N(R₈)₂, —C(═O)N(R₈)₂, —N(R₈)C(═O)R₉, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl. In another embodiment is a compound of Formula (I) wherein n is 0 and R₄ is H, —CN, —OH, —OCF₃, substituted or unsubstituted C₁-C₆alkyl, or substituted or unsubstituted C₁-C₆alkoxy. In another embodiment is a compound of Formula (I) wherein n is 0 and R₄ is H, —OCF₃, substituted or unsubstituted C₁-C₆alkyl, or substituted or unsubstituted C₁-C₆alkoxy.

In another embodiment is a compound of Formula (I) wherein n is 1, R₁ is Cl, and R₄ is Cl. In another embodiment is a compound of Formula (I) wherein n is 1, R₁ is —CH₃, and R₄ is Cl. In another embodiment is a compound of Formula (I) wherein n is 1, R₁ is —OCH₃, and R₄ is Cl. In another embodiment is a compound of Formula (I) wherein n is 1, R₁ is —CF₃, and R₄ is Cl. In another embodiment is a compound of Formula (I) wherein n is 1, R₁ is —OCF₃, and R₄ is Cl. In another embodiment is a compound of Formula (I) wherein n is 1, R₁ is Cl, and R₄ is F. In another embodiment is a compound of Formula (I) wherein n is 1, R₁ is —CH₃, and R₄ is F. In another embodiment is a compound of Formula (I) wherein n is 1, R₁ is —OCH₃, and R₄ is F. In another embodiment is a compound of Formula (I) wherein n is 1, R₁ is —CF₃, and R₄ is F. In another embodiment is a compound of Formula (I) wherein n is 1, R₁ is —OCF₃, and R₄ is F. In another embodiment is a compound of Formula (I) wherein n is 1, R₁ is Cl, and R₄ is H. In another embodiment is a compound of Formula (I) wherein n is 1, R₁ is —CH₃, and R₄ is H. In another embodiment is a compound of Formula (I) wherein n is 1, R₁ is —OCH₃, and R₄ is H. In another embodiment is a compound of Formula (I) wherein n is 1, R₁ is —CF₃, and R₄ is H. In another embodiment is a compound of Formula (I) wherein n is 1, R₁ is —OCF₃, and R₄ is H. In another embodiment is a compound of Formula (I) wherein n is 1, R₁ is Cl, and R₄ is —OCH₃. In another embodiment is a compound of Formula (I) wherein n is 1, R₁ is —CH₃, and R₄ is —OCH₃. In another embodiment is a compound of Formula (I) wherein n is 1, R₁ is —OCH₃, and R₄ is —OCH₃. In another embodiment is a compound of Formula (I) wherein n is 1, R₁ is —CF₃, and R₄ is —OCH₃. In another embodiment is a compound of Formula (I) wherein n is 1, R₁ is —OCF₃, and R₄ is —OCH₃. In another embodiment is a compound of Formula (I) wherein n is 1, R₁ is Cl, and R₄ is —OCF₃. In another embodiment is a compound of Formula (I) wherein n is 1, R₁ is —CH₃, and R₄ is —OCF₃. In another embodiment is a compound of Formula (I) wherein n is 1, R₁ is —OCH₃, and R₄ is —OCF₃. In another embodiment is a compound of Formula (I) wherein n is 1, R₁ is —CF₃, and R₄ is —OCF₃. In another embodiment is a compound of Formula (I) wherein n is 1, R₁ is —OCF₃, and R₄ is —OCF₃.

In another embodiment is a compound of Formula (I) wherein R₆ is —(C(R₁₄)(R₁₅))ₘN(R₁₁)(R₁₂) and m is 2-5. In another embodiment is a compound of Formula (I) wherein R₆ is —(C(R₁₄)(R₁₅))ₘN(R₁₁)(R₁₂), m is 2, and each R₁₄ and R₁₅ are H. In another embodiment is a compound of Formula (I) wherein R₆ is —(CH₂)ₘN(R₁₁)(R₁₂), m is 2, and R₁₁ and R₁₂ are each H. In another embodiment is a compound of Formula (I) wherein R₆ is —(CH₂)ₘN(R₁₁)(R₁₂), m is 2, R₁₁ is substituted or unsubstituted C₁-C₆alkyl, and R₁₁ is H. In another embodiment is a compound of Formula (I) wherein R₆ is —(CH₂)ₘN(R₁₁)(R₁₂), m is 2, R₁₁ is CH₃, and R₁₁ is H. In another embodiment is a compound of Formula (I) wherein R₆ is —(CH₂)ₘN(R₁₁)(R₁₂), m is 2, R₁₁ is substituted or unsubstituted C₁-C₆alkyl, and R₁₁ is substituted or unsubstituted C₁-C₆alkyl. In another embodiment is a compound of Formula (I) wherein R₆ is —(CH₂)ₘN(R₁₁)(R₁₁), m is 2, R₁ is CH₃, and R₁₁ is CH₃. In another embodiment is a compound of Formula (I) wherein R₆ is —(CH₂)ₘN(R₁₁)(R₂), m is 2, and R₁₁ and R₁₂ are taken together to form a substituted or unsubstituted 5-, 6-, 7-, or 8-membered heterocyclic ring. In another embodiment is a compound of Formula (I) wherein R₆ is —(CH₂)ₘN(R₁₁)(R₁₂), m is 2, and R₁₁ and R₁₂ are taken together to form a substituted or unsubstituted 5-membered heterocyclic ring. In another embodiment is a compound of Formula (I) wherein R₆ is —(CH₂)ₘN(R₁₁)(R₁₂), m is 2, and R₁₁ and R₁₂ are taken together to form a substituted or unsubstituted 6-membered heterocyclic ring.

In another embodiment is a compound of Formula (I) wherein R₆ is —(C(R₁₄)(R₁₅))ₘN(R₁₁)(R₁₁). In another embodiment is a compound of Formula (I) wherein R₆ is —(C(R₁₄)(R₁₅))ₘN(R₁₁)(R₁₂), m is 3, and each R₁₄ and R₁₅ are H. In another embodiment is a compound of Formula (I) wherein R₆ is —(CH₂)ₘN(R₁₁)(R₁₂), m is 3, and R₁₁ and R₁₂ are each H. In another embodiment is a compound of Formula (I) wherein R₆ is —(CH₂)ₘN(R₁₁)(R₁₂), m is 3, R₁₁ is substituted or unsubstituted C₁-C₆alkyl, and R₁₁ is H. In another embodiment is a compound of Formula (I) wherein R₆ is —(CH₂)ₘN(R₁₁)(R₁₂), m is 3, R₁₁ is CH₃, and R₁₁ is H. In another embodiment is a compound of Formula (I) wherein R₆ is —(CH₂)ₘN(R₁₁)(R₁₂), m is 3, R₁₁ is substituted or unsubstituted C₁-C₆alkyl, and R₁₁ is substituted or unsubstituted C₁-C₆alkyl. In another embodiment is a compound of Formula (I) wherein R₆ is —(CH₂)ₘN(R₁₁)(R₁₂), m is 3, R₁₁ is CH₃, and R₁₁ is CH₃. In another embodiment is a compound of Formula (I) wherein R₆ is —(CH₂)ₘN(R₁₁)(R₁₂), m is 3, and R₁₁ and R₁₂ are taken together to form a substituted or unsubstituted 5-, 6-, 7-, or 8-membered heterocyclic ring. In another embodiment is a compound of Formula (I) wherein R₆ is —(CH₂)ₘN(R₁₁)(R₁₂), m is 3, and R₁₁ and R₁₂ are taken together to form a substituted or unsubstituted 5-membered heterocyclic ring. In another embodiment is a compound of Formula (I) wherein R₆ is —(CH₂)ₘN(R₁₁)(R₁₂), m is 3, and R₁₁ and R₁₂ are taken together to form a substituted or unsubstituted 6-membered heterocyclic ring.

In another embodiment is a compound of Formula (I) wherein R₁₀ is H. In another embodiment is a compound of Formula (I) wherein R₁₀ is C₁-C₄alkyl. In another embodiment is a compound of Formula (I) wherein R₁₀ is —CH₃. In another embodiment is a compound of Formula (I) wherein R₁₀ is —CH₂CH₃.

In another embodiment is a compound of Formula (I) wherein R₅ is halogen. In another embodiment is a compound of Formula (I) wherein R₅ is —CF₃. In another embodiment is a compound of Formula (I) wherein R₅ is substituted or unsubstituted C₁-C₆alkyl. In another embodiment is a compound of Formula (I) wherein R₅ is —CH₃. In another embodiment is a compound of Formula (I) wherein R₅ is —CH₂CH₃. In another embodiment is a compound of Formula (I) wherein R₅ is substituted or unsubstituted C₁-C₆heteroalkyl. In another embodiment is a compound of Formula (I) wherein R₅ is substituted or unsubstituted C₂-C₇heterocycloalkyl. In another embodiment is a compound of Formula (I) wherein R₅ is substituted or unsubstituted C₃-C₈cycloalkyl. In another embodiment is a compound of Formula (I) wherein R₅ is substituted or unsubstituted C₆-C₁₀aryl. In another embodiment is a compound of Formula (I) wherein R₅ is substituted or unsubstituted C₂-C₇heteroaryl.

In some embodiments the compound of Formula I is 1-(3,4-dichlorophenyl)-3-(4-(3-(dimethylamino)propylamino)-6-methylpyrimidin-2-yl)urea having the structure:

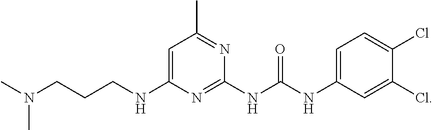 [SMILES: Cc1cc(NCCCN(C)C)nc(NC(=O)Nc2ccc(Cl)c(Cl)c2)n1]

In some embodiments described herein is a pharmaceutical composition comprising 1) a first therapeutic agent, 2) a second therapeutic agent, and 3) at least one pharmaceutically acceptable excipient, wherein the first therapeutic agent is a compound of Formula (II), or a pharmaceutically acceptable salt, solvate, or prodrug thereof, having the structure:

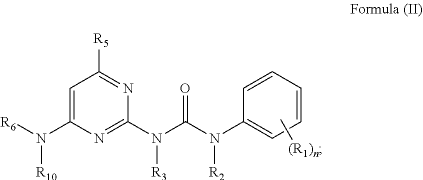

wherein:
   each R₁ is independently halogen, —CN, —NO₂, —OH, —OCF₃, —OCH₂F, —OCF₂H, —CF₃, —SR₈, —N(R₈)S(═O)₂R₉, —S(═O)₂N(R₈)₂, —S(═O)R₉, —S(═O)₂R₉, —C(═O)R₉, —CO₂R₈, —N(R₈)₂, —C(═O)N(R₈)₂, —N(R₈)C(═O)R₉, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl; or two R₁ are taken together to form a substituted or unsubstituted heterocyclic ring or a substituted or unsubstituted carbocyclic ring;
   R₂ and R₃ are each independently H, —CN, C₁-C₄alkyl, C₃-C₆cycloalkyl, or C₂-C₇heterocycloalkyl; or R₂ and R₃ are taken together to form a 5- or 6-membered heterocyclic ring;
   R₅ is halogen, —CN, —OH, —CF₃, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl;
   R₆ is substituted or unsubstituted C₂-C₇heterocycloalkyl, substituted or unsubstituted C₃-C₈cycloalkyl, substituted or unsubstituted C₂-C₇heteroaryl, —(C(R₁₄)(R₁₅))ₘR₂₁, or

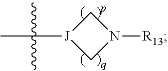

J is C(H);
R₁₃ is H, substituted or unsubstituted C₁-C₆alkyl, substituted or unsubstituted C₂-C₇heterocycloalkyl, substituted or unsubstituted C₃-C₈cycloalkyl, substituted or unsubstituted —C₁-C₄alkylC₆-C₁₀aryl, or substituted or unsubstituted —C₁-C₄alkylC₂-C₇heteroaryl;
each R₁₄ and R₁₅ are each independently H, or substituted or unsubstituted C₁-C₆alkyl; or
R₁₄ and R₁₅ are taken together to form a 4-, 5-, 6-membered cycloalkyl ring;
R₂₁ is halogen, —CN, —NO₂, —OH, —OCF₃, —OCH₂F, —OCF₂H, —CF₃, —SR₂₂, —N(R₂₂)S(═O)₂R₂₃, —S(═O)₂N(R₂₂)₂, —S(═O)R₂₃, —S(═O)₂R₂₃, —C(═O)R₂₃, —CO₂R₂₂, —C(═O)N(R₂₂)₂, —N(R₂₂)C(═O)R₂₃, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted C₆-C₁₀aryl, or substituted or unsubstituted C₂-C₇heteroaryl;
each R₂₂ is independently H, or substituted or unsubstituted C₁-C₆alkyl;
R₂₃ is substituted or unsubstituted C₁-C₆alkyl;
each R₈ is independently H, or substituted or unsubstituted C₁-C₆alkyl;
each R₉ is independently substituted or unsubstituted C₁-C₆alkyl;
R₁₀ is H or unsubstituted C₁-C₄alkyl;
m is 2-6;
n is 0-5;
p is 1-3;
q is 1-3; or
a pharmaceutically acceptable salt, solvate, or prodrug thereof.

In another embodiment is a compound of Formula (II) wherein R₆ is substituted or unsubstituted C₃-C₈cycloalkyl. In another embodiment is a compound of Formula (II) wherein R₆ is substituted or unsubstituted cyclopropyl. In another embodiment is a compound of Formula (II) wherein R₆ is substituted or unsubstituted cyclobutyl. In another embodiment is a compound of Formula (II) wherein R₆ is substituted or unsubstituted cyclopentyl. In another embodiment is a compound of Formula (II) wherein R₆ is substituted or unsubstituted cyclohexyl.

In another embodiment is a compound of Formula (II) wherein R₆ is substituted or unsubstituted C₂-C₇heteroaryl. In another embodiment is a compound of Formula (II) wherein R₆ is substituted or unsubstituted furanyl, thiophenyl, pyrrolyl, pyridyl, oxazolyl, thiazolyl, imidazolyl, isoxazolyl, isothiazolyl, pyrazolyl, pyridazinyl, pyrimidinyl, pyrazinyl, oxadiazolyl, thiadiazolyl, triazolyl, indolyl, benzothiophenyl, benzoxazolyl, benzothiazolyl, benzimidazolyl, benzoxadiazolyl, benzothiadiazolyl, benzotriazolyl, pyrazolopyridinyl, imidazopyridinyl, pyrrolopyridinyl, pyrrolopyrimidinyl, indolizinyl, purinyl, furopyridinyl, thienopyridinyl, furopyrrolyl, furofuranyl, thienofuranyl, 1,4-dihydropyrrolopyrrolyl, thienopyrrolyl, thienothiophenyl, quinolinyl, isoquinolinyl, quinoxalinyl, furopyrazolyl, thienopyrazolyl, selenophenyl, selenazolyl, or benzoisoxazolyl. In another embodiment is a compound of Formula (II) wherein R₆ is substituted or unsubstituted pyridyl. In another embodiment is a compound of Formula (II) wherein R₆ is substituted pyridyl. In another embodiment is a compound of Formula (II) wherein R₆ is unsubstituted pyridyl.

In another embodiment is a compound of Formula (II) wherein R₆ is —(C(R₁₄)(R₁₅))ₘR₂₁. In another embodiment is a compound of Formula (II) wherein R₆ is —(C(R₁₄)(R₁₅))ₘR₂₁, and each R₁₄ and R₁₅ are H. In another embodiment is a compound of Formula (II) wherein R₆ is —(C(R₁₄)(R₁₅))ₘR₂₁, m is 2, and each R₁₄ and R₁₅ are H. In another embodiment is a compound of Formula (II) wherein R₆ is —(CH₂)ₘR₂₁, m is 2, and R₂₁ is substituted or unsubstituted C₂-C₇heterocycloalkyl. In another embodiment is a compound of Formula (II) wherein R₆ is —(CH₂)ₘR₂₁, m is 2, and R₂₁ is substituted or unsubstituted C₃-C₈cycloalkyl. In another embodiment is a compound of Formula (II) wherein R₆ is —(CH₂)ₘR₂₁, m is 2, and R₂₁ is substituted C₆-C₁₀aryl. In another embodiment is a compound of Formula (II) wherein R₆ is —(CH₂)ₘR₂₁, m is 2, and R₂₁ is substituted phenyl. In another embodiment is a compound of Formula (II) wherein R₆ is —(CH₂)ₘR₂₁, m is 2, and R₂₁ is substituted or unsubstituted C₂-C₇heteroaryl. In another embodiment is a compound of Formula (II) wherein R₆ is —(CH₂)ₘR₂₁, m is 2, and R₂₁ is —OH. In another embodiment is a compound of Formula (II) wherein R₆ is —(CH₂)ₘR₂₁, m is 2, and R₂₁ is —N(R₂₂)S(═O)₂R₂₃. In another embodiment is a compound of Formula (II) wherein R₆ is —(CH₂)ₘR₂₁, m is 2, and R₂₁ is —N(R₂₂)C(═O)R₂₃. In another embodiment is a compound of Formula (II) wherein R₆ is —(CH₂)ₘR₂₁, m is 2, and R₂₁ is substituted or unsubstituted C₁-C₆alkoxy. In another embodiment of the aforementioned embodiments, is a compound of Formula (II) wherein each R₂₂ is independently H or unsubstituted C₁-C₆alkyl; and R₂₃ is unsubstituted C₁-C₆alkyl.

In another embodiment is a compound of Formula (II) wherein R₆ is —(C(R₁₄)(R₁₅))ₘR₂₁, m is 3, and each R₁₄ and R₁₅ are H. In another embodiment is a compound of Formula (II) wherein R₆ is —(CH₂)ₘR₂₁, m is 3, and R₂₁ is substituted or unsubstituted C₂-C₇heterocycloalkyl. In another embodiment is a compound of Formula (II) wherein R₆ is —(CH₂)ₘR₂₁, m is 3, and R₂₁ is substituted or unsubstituted C₃-C₈cycloalkyl. In another embodiment is a compound of Formula (II) wherein R₆ is —(CH₂)ₘR₂₁, m is 3, and R₂₁ is substituted C₆-C₁₀aryl. In another embodiment is a compound of Formula (II) wherein R₆ is —(CH₂)ₘR₂₁, m is 3, and R₂₁ is substituted phenyl. In another embodiment is a compound of Formula (II) wherein R₆ is —(CH₂)ₘR₂₁, m is 3, and R₂₁ is substituted or unsubstituted C₂-C₇heteroaryl. In another embodiment is a compound of Formula (II) wherein R₆ is —(CH₂)ₘR₂₁, m is 3, and R₂₁ is —OH. In another embodiment is a compound of Formula (II) wherein R₆ is —(CH₂)ₘR₂₁, m is 3, and R₂₁ is —N(R₂₂)S(═O)₂R₂₃. In another embodiment is a compound of Formula (II) wherein R₆ is —(CH₂)ₘR₂₁, m is 3, and R₂₁ is —N(R₂₂)C(═O)R₂₃. In another embodiment is a compound of Formula (II) wherein R₆ is —(CH₂)ₘR₂₁, m is 3, and R₂₁ is substituted or unsubstituted C₁-C₆alkoxy. In another embodiment of the aforementioned embodiments, is a compound of Formula (II) wherein each R₂₂ is independently H or unsubstituted C₁-C₆alkyl; and R₂₃ is unsubstituted C₁-C₆alkyl.

In another embodiment is a compound of Formula (II) wherein R₆ is substituted or unsubstituted C₂-C₇heterocycloalkyl. In another embodiment is a compound of Formula (II) wherein R₆ is

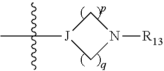

and J is C(H). In another embodiment is a compound of Formula (II) wherein R₆ is

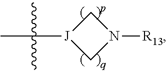

J is C(H), p is 1, and q is 1. In another embodiment is a compound of Formula (II) wherein R₆ is

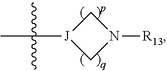

J is C(H), p is 2, and q is 1. In another embodiment is a compound of Formula (II) wherein R₆ is

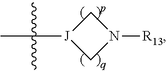

J is C(H), p is 3, and q is 1. In another embodiment is a compound of Formula (II) wherein R₆ is

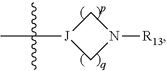

J is C(H), p is 2, and q is 2. In another embodiment is a compound of Formula (II) wherein R₆ is

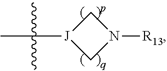

J is C(H), p is 1, q is 1, and R₁₃ is substituted or unsubstituted C₁-C₆alkyl. In another embodiment is a compound of Formula (II) wherein R₆ is

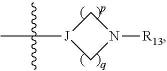

J is C(H), p is 2, q is 1, and R₁₃ is substituted or unsubstituted C₁-C₆alkyl. In another embodiment is a compound of Formula (II) wherein R₆ is

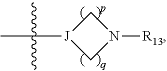

J is C(H), p is 3, q is 1, and RU is substituted or unsubstituted C₁-C₆alkyl. In another embodiment is a compound of Formula (II) wherein R₆ is

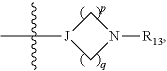

J is C(H), p is 2, q is 2, and R₁₃ is substituted or unsubstituted C₁-C₆alkyl.

In another embodiment is a compound of Formula (II) wherein R₅ is halogen. In another embodiment is a compound of Formula (II) wherein R₅ is —CF₃. In another embodiment is a compound of Formula (II) wherein R₈ is substituted or unsubstituted C₁-C₆alkyl. In another embodiment is a compound of Formula (II) wherein R₈ is —CH₃. In another embodiment is a compound of Formula (II) wherein R₅ is —CH₂CH₃. In another embodiment is a compound of Formula (II) wherein R₈ is substituted or unsubstituted C₁-C₆heteroalkyl. In another embodiment is a compound of Formula (II) wherein R₈ is substituted or unsubstituted C₂-C₇heterocycloalkyl. In another embodiment is a compound of Formula (II) wherein R₅ is substituted or unsubstituted C₃-C₈cycloalkyl. In another embodiment is a compound of Formula (II) wherein R₅ is substituted or unsubstituted C₆-C₁₀aryl. In another embodiment is a compound of Formula (II) wherein R₅ is substituted or unsubstituted C₂-C₇heteroaryl.

In another embodiment is a compound of Formula (II) wherein R₂ and R₃ are each independently H, —CN, C₁-C₄alkyl, C₃-C₆cycloalkyl, or C₂-C₇heterocycloalkyl. In another embodiment is a compound of Formula (II) wherein R₂ and R₃ are each H.

In another embodiment is a compound of Formula (II) wherein R₂ and R₃ are each independently H, —CN, C₁-C₄alkyl, C₃-C₆cycloalkyl, or C₂-C₇heterocycloalkyl; and at least one of R₂ and R₃ is not H. In another embodiment is a compound of Formula (II) wherein R₂ is H, and R₃ is C₁-C₄alkyl. In another embodiment is a compound of Formula (II) wherein R₂ is H, and R₃ is CH₃. In another embodiment is a compound of Formula (II) wherein R₂ is H, and R₃ is C₃-C₆cycloalkyl. In another embodiment is a compound of Formula (II) wherein R₂ is H, and R₃ is cyclopropyl. In another embodiment is a compound of Formula (II) wherein R₂ is H, and R₃ is cyclopentyl. In another embodiment is a compound of Formula (II) wherein R₂ is CH₃, and R₃ is CH₃. In another embodiment is a compound of Formula (II) wherein R₂ is C₁-C₄alkyl, and R₃ is H. In another embodiment is a compound of Formula (II) wherein R₂ is CH₃, and R₃ is H. In another embodiment is a compound of Formula (II) wherein R₂ is C₃-C₆cycloalkyl, and R₃ is H. In another embodiment is a compound of Formula (II) wherein R₂ is cyclopropyl, and R₃ is H. In another embodiment is a compound of Formula (II) wherein R₂ is cyclopentyl, and R₃ is H.

In another embodiment is a compound of Formula (II) wherein R₂ and R₃ are taken together to form a 5- or 6-membered heterocyclic ring. In another embodiment is a compound of Formula (II) wherein R₂ and R₃ are taken together to form a 5-membered heterocyclic ring. In another embodiment is a compound of Formula (II) wherein R₂ and R₃ are taken together to form 6-membered heterocyclic ring.

In another embodiment is a compound of Formula (II) wherein n is 0.

In another embodiment is a compound of Formula (II) wherein each R₁ is independently halogen, —CN, —NO₂, —OH, —OCF₃, —OCH₂F, —OCF₂H, —SR₈, —N(R₈)S(═O)₂R₉, —S(═O)₂N(R₈)₂, —S(═O)R₉, —S(═O)₂R₉, —C(═O)R₉, —CO₂R₈, —N(R₈)₂, —C(═O)N(R₈)₂, —N(R₈)C(═O)R₉, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl. In another embodiment is a compound of Formula (II) wherein each R₁ is independently halogen, —CN, —OH, substituted or unsubstituted C₁-C₆alkyl, or substituted or unsubstituted C₁-C₆alkoxy. In another embodiment is a compound of Formula (II) wherein each R₁ is independently halogen, —CN, —OCF₃, —OCH₂F, —OCF₂H, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl. In another embodiment is a compound of Formula (II) wherein each R₁ is independently halogen, —CN, —OCF₃, —OCH₂F, —OCF₂H, substituted or unsubstituted C₁-C₆alkyl, or substituted or unsubstituted C₁-C₆alkoxy. In another embodiment is a compound of Formula (II) wherein each R₁ is independently halogen, —CN, —OCF₃, —OCH₂F, —OCF₂H, substituted or unsubstituted C₁-C₆alkyl, or substituted or unsubstituted C₁-C₆alkoxy, and n is 3. In another embodiment is a compound of Formula (II) wherein each R₁ is independently halogen, —CN, —OCF₃, —OCH₂F, —OCF₂H, substituted or unsubstituted C₁-C₆alkyl, or substituted or unsubstituted C₁-C₆alkoxy, and n is 2. In another embodiment is a compound of Formula (II) wherein n is 3, and each R₁ is independently halogen. In another embodiment is a compound of Formula (II) wherein n is 2, and each R₁ is independently halogen. In another embodiment is a compound of Formula (II) wherein n is 2, and each R₁ is independently F or Cl. In another embodiment is a compound of Formula (II) wherein n is 2, and each R₁ is F. In another embodiment is a compound of Formula (II) wherein n is 2, and each R₁ is independently Cl. In another embodiment is a compound of Formula (II) wherein n is 1, and R₁ is halogen. In another embodiment is a compound of Formula (II) wherein n is 1, and R₁ is F. In another embodiment is a compound of Formula (II) wherein n is 1, and R₁ is Cl. In another embodiment is a compound of Formula (II) wherein n is 1, and R₁ is substituted or unsubstituted C₁-C₆alkyl. In another embodiment is a compound of Formula (II) wherein n is 1, and R₁ is CH₃. In another embodiment is a compound of Formula (II) wherein n is 1, and R₁ is substituted or unsubstituted C₁-C₆alkoxy. In another embodiment is a compound of Formula (II) wherein n is 1, and R₁ is —OCH₃. In another embodiment is a compound of Formula (II) wherein n is 1, and R₁ is —OCF₃. In another embodiment is a compound of Formula (II) wherein n is 1, and R₁ is —OCF₂H.

In another embodiment is a compound selected from.

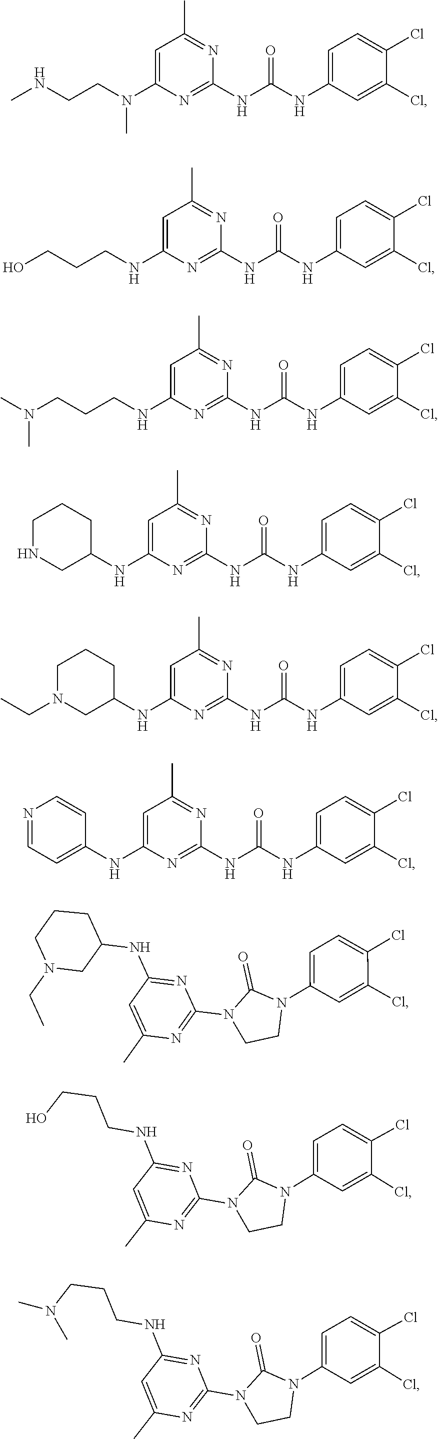

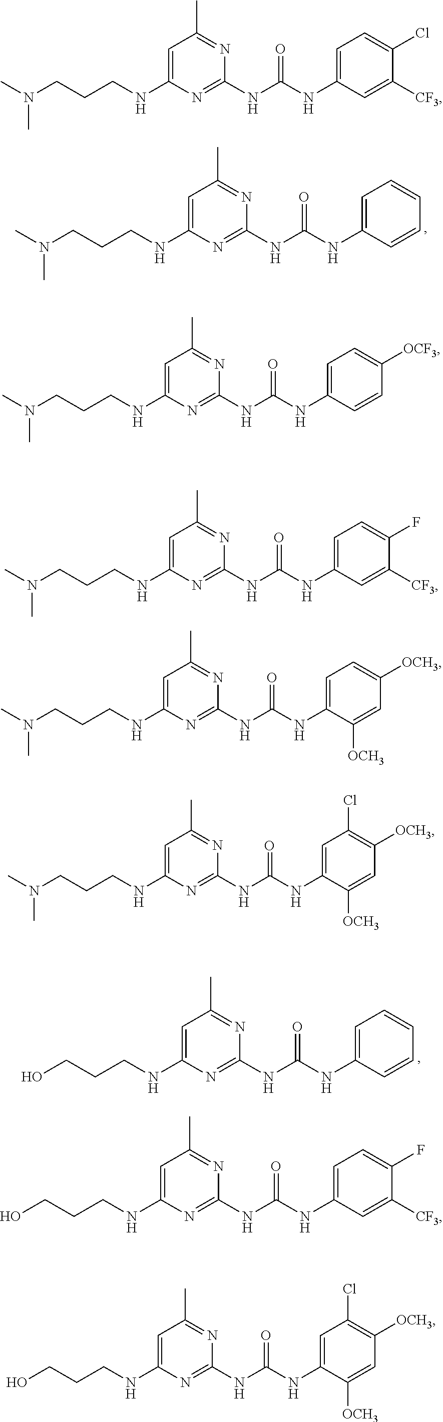

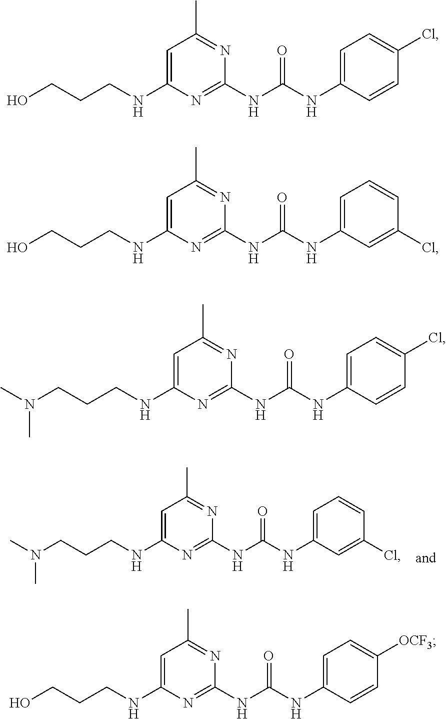 [SMILES: Cc1cc(NCCCO)nc(NC(=O)Nc2ccc(Cl)cc2)n1 ; Cc1cc(NCCCO)nc(NC(=O)Nc2cccc(Cl)c2)n1 ; Cc1cc(NCCCN(C)C)nc(NC(=O)Nc2ccc(Cl)cc2)n1 ; Cc1cc(NCCCN(C)C)nc(NC(=O)Nc2cccc(Cl)c2)n1 ; Cc1cc(NCCCO)nc(NC(=O)Nc2ccc(OC(F)(F)F)cc2)n1]

or a pharmaceutically acceptable salt, solvate, or prodrug thereof.

Any combination of the groups described above for the various variables is contemplated herein.

Throughout the specification, groups and substituents thereof can be chosen to provide stable moieties and compounds.

### EGFR Inhibitors:

Disclosed herein are EGFR inhibitors used in combination with a first agent that is a compound of Formula (I) or (II). In some embodiments, the EGFR inhibitor is selected from gefitinib, erlotinib, lapatinib, cetuximab, panitumumab, vandetanib, necitumumab, osimertinib, tesevatinib, pelitinib, rocilitinib, and JNJ2887.

ATM Inhibitors:

Disclosed herein are ATM inhibitors used in combination with a first agent that is a compound of Formula (I) or (II). In some embodiments, the ATM inhibitor is selected from KU-55933, KU-60019, wortmannin, torin 2, CP-466722, and CGK-733.

ATR Inhibitors:

Disclosed herein are ATR inhibitors used in combination with a first agent that is a compound of Formula (I) or (II). In some embodiments, the ATR inhibitor is selected from dactolisib, VE-821, VE-822, ETP-46464, CGK-733, AZ-20, and AZD-6738.

PARP Inhibitors:

Disclosed herein are PARP inhibitors used in combination with a first agent that is a compound of Formula (I) or (II). In some embodiments, the PARP inhibitor is selected from niraparib, iniparib, talazoparib, veliparib, oloparib, rucaparib, CEP-9722, E7106, and BGB-290.

CDK4/6 Inhibitors:

Disclosed herein are CDK4/6 inhibitors used in combination with a first agent that is a compound of Formula (I) or (II). In some embodiments, the CDK4/6 inhibitor is selected from palbociclib, abemaciclib, P1446A-05, ribociclib, R547, LY2835219, alvocidib, PHA-793887, P276-00, AT7519, milciclib, SU9516, BMS-265246, JNJ-7706621, SNS-032, LDC000067, and LEE011.

Chk1 Inhibitors:

Disclosed herein are Chk1 inhibitors used in combination with a first agent that is a compound of Formula (I) or (II). In some embodiments, the Chk1 inhibitor is selected from CHIR-124, PF-477736, MK-8776, LY2603618, and AZD7762.

JAK2 Inhibitors:

Disclosed herein are JAK2 inhibitors used in combination with a first agent that is a compound of Formula (I) or (II). In some embodiments, the JAK2 inhibitor is selected from ruxolitnib, tofacitinib, baricitinib, filgotinib, gandotinib, lestaurtinib, momelotinib, pacritinib, upadacitinib, fedratinib, cerdulatinib, AZD1480, AZ 960, NVP-BSK805, CEP-33779, TG101209, WP1066, AG-490, GLPG0634, Go6976, LY2784544, and AT9283.

mTOR Inhibitors:

Disclosed herein are mTOR inhibitors used in combination with a first agent that is a compound of Formula (I) or (II). In some embodiments, the mTOR inhibitor is selected from rapamycin, temsirolimus, everolimus, ridaforolimus, torkinib, voxtalisib, torin 1, torin 2, omipalisib, apitolisib, gedatolisib, vistusertib, AZD8055, SF2523, CZ415, LY3023414, PI-103, KU-0063794, INK-128, OSI-027, PF-04691502, WYE-354, WYE-125132, WYE-687, BGT226, and WAY-600.

STAT3 Inhibitors:

Disclosed herein are STAT3 inhibitors used in combination with a first agent that is a compound of Formula (I) or (II). In some embodiments, the STAT3 inhibitor is selected from S3I-201, stattic, niclosamide, napabucasin, cryptotanshinone, HO-3867, SH-4-54, LY5, C₁₈₈-9, LLL12, and STX-0119.

Further Forms of Compounds

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by the forming diastereomeric and separation by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

In some situations, compounds may exist as tautomers. All tautomers are included within the formulas described herein.

The methods and compositions described herein include the use of amorphous forms as well as crystalline forms (also known as polymorphs). The compounds described herein may be in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In some embodiments, compounds described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

To produce a prodrug, a pharmaceutically active compound is modified such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. In some embodiments, by virtue of knowledge of pharmacodynamic processes and drug metabolism in vivo, once a pharmaceutically active compound is determined, prodrugs of the compound are designed. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Saulnier et al., (1994), Bioorganic and Medicinal Chemistry Letters, Vol. 4, p. 1985; Rooseboom et al., Pharmacological Reviews, 56:53-102, 2004; Miller et al., J. Med. Chem. Vol. 46, no. 24, 5097-5116, 2003; Aesop Cho, “Recent Advances in Oral Prodrug Discovery”, Annual Reports in Medicinal Chemistry, Vol. 41, 395-407, 2006).

Prodrug forms of the herein described compounds, wherein the prodrug is metabolized in vivo to produce a compound of Formula (I) or (II) as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. Prodrugs may be designed as reversible drug derivatives, for use as modifiers to enhance drug transport to site-specific tissues. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure).

Sites on the aromatic ring portion of compounds described herein can be susceptible to various metabolic reactions, therefore incorporation of appropriate substituents on the aromatic ring structures, such as, by way of example only, halogens can reduce, minimize, or eliminate this metabolic pathway.

The compounds described herein may be labeled isotopically (e.g. with a radioisotope) or by other means, including, but not limited to, the use of chromophores or fluorescent moieties, bioluminescent labels, photoactivatable labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine and chlorine, such as, for example, ²H, ³H, ¹³C, ¹⁴C, ¹⁵N, ¹⁸O, ¹⁷O, ³⁵S, ¹⁸F, ³⁶Cl, respectively. Certain isotopically-labeled compounds described herein, for example those into which radioactive isotopes such as ³H and ¹⁴C are incorporated, are useful in drug and/or substrate tissue distribution assays. Further, substitution with isotopes such as deuterium, i.e., ²H, can afford certain therapeutic advantages resulting from greater metabolic stability, such as, for example, increased in vivo half-life or reduced dosage requirements. Generally, in chemical compounds with an H atom, the H atom actually represents a mixture of H and D, with about 0.015% being D. In some embodiments, deuterium-enriched compounds described herein are achieved by either exchanging protons with deuterium or via starting materials and/or intermediates enriched with deuterium.

In additional or further embodiments, the compounds described herein are metabolized upon administration to an organism in need to produce a metabolite that is then used to produce a desired effect, including a desired therapeutic effect.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

In some embodiments, compounds described herein, such as compounds of Formula (I) or (II), are in various forms, including but not limited to, amorphous forms, milled forms and nano-particulate forms. In addition, compounds described herein include crystalline forms, also known as polymorphs. Polymorphs include the different crystal packing arrangements of the same elemental composition of a compound. Polymorphs usually have different X-ray diffraction patterns, melting points, density, hardness, crystal shape, optical properties, stability, and solubility. Various factors such as the recrystallization solvent, rate of crystallization, and storage temperature may cause a single crystal form to dominate.

The screening and characterization of the pharmaceutically acceptable salts, polymorphs and/or solvates may be accomplished using a variety of techniques including, but not limited to, thermal analysis, x-ray diffraction, spectroscopy, vapor sorption, and microscopy. Thermal analysis methods address thermo chemical degradation or thermo physical processes including, but not limited to, polymorphic transitions, and such methods are used to analyze the relationships between polymorphic forms, determine weight loss, to find the glass transition temperature, or for excipient compatibility studies. Such methods include, but are not limited to, Differential Scanning Calorimetry (DSC), Modulated Differential Scanning Calorimetry (MDCS), Thermogravimetric analysis (TGA), and Thermogravi-metric and Infrared analysis (TG/IR). X-ray diffraction methods include, but are not limited to, single crystal and powder diffractometers and synchrotron sources. The various spectroscopic techniques used include, but are not limited to, Raman, FTIR, UV-VIS, and NMR (liquid and solid state). The various microscopy techniques include, but are not limited to, polarized light microscopy, Scanning Electron Microscopy (SEM) with Energy Dispersive X-Ray Analysis (EDX), Environmental Scanning Electron Microscopy with EDX (in gas or water vapor atmosphere), IR microscopy, and Raman microscopy.

Throughout the specification, groups and substituents thereof can be chosen to provide stable moieties and compounds.

### Combination Treatment

In some embodiments, the compounds disclosed herein are used in combination for the treatment of a disease, disorder or condition in a mammal that would benefit from combined OLIG2 modulation and EGFR inhibition. In some embodiments, the compounds disclosed herein are used in combination for the treatment of a disease, disorder or condition in a mammal that would benefit from combined OLIG2 modulation and ATM inhibition. In some embodiments, the compounds disclosed herein are used in combination for the treatment of a disease, disorder or condition in a mammal that would benefit from combined OLIG2 modulation and ATR inhibition. In some embodiments, the compounds disclosed herein are used in combination for the treatment of a disease, disorder or condition in a mammal that would benefit from combined OLIG2 modulation and PARP inhibition. In some embodiments, the compounds disclosed herein are used in combination for the treatment of a disease, disorder or condition in a mammal that would benefit from combined OLIG2 modulation and CDK4/6 inhibition. In some embodiments, the compounds disclosed herein are used in combination for the treatment of a disease, disorder or condition in a mammal that would benefit from combined OLIG2 modulation and Chk1 inhibition. In some embodiments, the compounds disclosed herein are used in combination for the treatment of a disease, disorder or condition in a mammal that would benefit from combined OLIG2 modulation and JAK2 inhibition. In some embodiments, the compounds disclosed herein are used in combination for the treatment of a disease, disorder or condition in a mammal that would benefit from combined OLIG2 modulation and mTOR inhibition. In some embodiments, the compounds disclosed herein are used in combination for the treatment of a disease, disorder or condition in a mammal that would benefit from combined OLIG2 modulation and STAT3 inhibition.

Disclosed herein is a method of treating cancer in a subject in need thereof comprising administering to the subject a therapeutically effective amount of a pharmaceutical composition comprising 1) a first therapeutic agent that is an OLIG2 modulator described herein, 2) a second therapeutic agent that is an EGFR inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (I) described herein, 2) a second therapeutic agent that is an EGFR inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (I) described herein, 2) a second therapeutic agent that is an EGFR inhibitor, and 3) at least one pharmaceutically acceptable excipient, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments is a method of treating cancer in a subject in need thereof comprising administering to the subject a therapeutically effective amount of a pharmaceutical composition comprising 1) a first therapeutic agent that is a compound of Formula (II) described herein, 2) a second therapeutic agent that is an EGFR inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (II) described herein, 2) a second therapeutic agent that is an EGFR inhibitor, and 3) at least one pharmaceutically acceptable excipient, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments of the aforementioned methods, the EGFR inhibitor is selected from gefitinib, erlotinib, lapatinib, cetuximab, panitumumab, vandetanib, necitumumab, osimertinib, tesevatinib, pelitinib, rocilitinib, and JNJ2887.

Disclosed herein is a method of treating cancer in a subject in need thereof comprising administering to the subject a therapeutically effective amount of a pharmaceutical composition comprising 1) a first therapeutic agent that is an OLIG2 modulator described herein, 2) a second therapeutic agent that is an ATM inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (I) described herein, 2) a second therapeutic agent that is an ATM inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (I) described herein, 2) a second therapeutic agent that is an ATM inhibitor, and 3) at least one pharmaceutically acceptable excipient, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments is a method of treating cancer in a subject in need thereof comprising administering to the subject a therapeutically effective amount of a pharmaceutical composition comprising 1) a first therapeutic agent that is a compound of Formula (II) described herein, 2) a second therapeutic agent that is an ATM inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (II) described herein, 2) a second therapeutic agent that is an ATM inhibitor, and 3) at least one pharmaceutically acceptable excipient, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments of the aforementioned methods, the ATM inhibitor is selected from KU-55933, KU-60019, wortmannin, torin 2, CP-466722, and CGK-733.

Disclosed herein is a method of treating cancer in a subject in need thereof comprising administering to the subject a therapeutically effective amount of a pharmaceutical composition comprising 1) a first therapeutic agent that is an OLIG2 modulator described herein, 2) a second therapeutic agent that is an ATR inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (I) described herein, 2) a second therapeutic agent that is an ATR inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (I) described herein, 2) a second therapeutic agent that is an ATR inhibitor, and 3) at least one pharmaceutically acceptable excipient, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments is a method of treating cancer in a subject in need thereof comprising administering to the subject a therapeutically effective amount of a pharmaceutical composition comprising 1) a first therapeutic agent that is a compound of Formula (II) described herein, 2) a second therapeutic agent that is an ATR inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (II) described herein, 2) a second therapeutic agent that is an ATR inhibitor, and 3) at least one pharmaceutically acceptable excipient, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments of the aforementioned methods, the ATR inhibitor is selected from dactolisib, VE-821, VE-822, ETP-46464, CGK-733, AZ-20, and AZD-6738.

Disclosed herein is a method of treating cancer in a subject in need thereof comprising administering to the subject a therapeutically effective amount of a pharmaceutical composition comprising 1) a first therapeutic agent that is an OLIG2 modulator described herein, 2) a second therapeutic agent that is a PARP inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (I) described herein, 2) a second therapeutic agent that is a PARP inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (I) described herein, 2) a second therapeutic agent that is a PARP inhibitor, and 3) at least one pharmaceutically acceptable excipient, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments is a method of treating cancer in a subject in need thereof comprising administering to the subject a therapeutically effective amount of a pharmaceutical composition comprising 1) a first therapeutic agent that is a compound of Formula (II) described herein, 2) a second therapeutic agent that is a PARP inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (II) described herein, 2) a second therapeutic agent that is a PARP inhibitor, and 3) at least one pharmaceutically acceptable excipient, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments of the aforementioned methods, the PARP inhibitor is selected from niraparib, iniparib, talazoparib, veliparib, oloparib, rucaparib, CEP-9722, E7106, and BGB-290.

Disclosed herein is a method of treating cancer in a subject in need thereof comprising administering to the subject a therapeutically effective amount of a pharmaceutical composition comprising 1) a first therapeutic agent that is an OLIG2 modulator described herein, 2) a second therapeutic agent that is a CDK4/6 inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (I) described herein, 2) a second therapeutic agent that is a CDK4/6 inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (I) described herein, 2) a second therapeutic agent that is a CDK4/6 inhibitor, and 3) at least one pharmaceutically acceptable excipient, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments is a method of treating cancer in a subject in need thereof comprising administering to the subject a therapeutically effective amount of a pharmaceutical composition comprising 1) a first therapeutic agent that is a compound of Formula (II) described herein, 2) a second therapeutic agent that is a CDK4/6 inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (II) described herein, 2) a second therapeutic agent that is a CDK4/6 inhibitor, and 3) at least one pharmaceutically acceptable excipient, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments of the aforementioned methods, the CDK4/6 inhibitor is selected from palbociclib, abemaciclib, P1446A-05, ribociclib, R547, LY2835219, alvocidib, PHA-793887, P276-00, AT7519, milciclib, SU9516, BMS-265246, JNJ-7706621, SNS-032, LDC000067, and LEE011.

Disclosed herein is a method of treating cancer in a subject in need thereof comprising administering to the subject a therapeutically effective amount of a pharmaceutical composition comprising 1) a first therapeutic agent that is an OLIG2 modulator described herein, 2) a second therapeutic agent that is a Chk1 inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (I) described herein, 2) a second therapeutic agent that is a Chk1 inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (I) described herein, 2) a second therapeutic agent that is a Chk1 inhibitor, and 3) at least one pharmaceutically acceptable excipient, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments is a method of treating cancer in a subject in need thereof comprising administering to the subject a therapeutically effective amount of a pharmaceutical composition comprising 1) a first therapeutic agent that is a compound of Formula (II) described herein, 2) a second therapeutic agent that is a Chk1 inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (II) described herein, 2) a second therapeutic agent that is a Chk1 inhibitor, and 3) at least one pharmaceutically acceptable excipient, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments of the aforementioned methods, the Chk1 inhibitor is selected from CHIR-124, PF-477736, MK-8776, LY2603618, and AZD7762.

Disclosed herein is a method of treating cancer in a subject in need thereof comprising administering to the subject a therapeutically effective amount of a pharmaceutical composition comprising 1) a first therapeutic agent that is an OLIG2 modulator described herein, 2) a second therapeutic agent that is a JAK2 inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (I) described herein, 2) a second therapeutic agent that is a JAK2 inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (I) described herein, 2) a second therapeutic agent that is a JAK2 inhibitor, and 3) at least one pharmaceutically acceptable excipient, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments is a method of treating cancer in a subject in need thereof comprising administering to the subject a therapeutically effective amount of a pharmaceutical composition comprising 1) a first therapeutic agent that is a compound of Formula (II) described herein, 2) a second therapeutic agent that is a JAK2 inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (II) described herein, 2) a second therapeutic agent that is a JAK2 inhibitor, and 3) at least one pharmaceutically acceptable excipient, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments of the aforementioned methods, the JAK2 inhibitor is selected from ruxolitnib, tofacitinib, baricitinib, filgotinib, gandotinib, lestaurtinib, momelotinib, pacritinib, upadacitinib, fedratinib, cerdulatinib, AZD1480, AZ 960, NVP-BSK805, CEP-33779, TG101209, WP1066, AG-490, GLPG0634, Go6976, LY2784544, and AT9283.

Disclosed herein is a method of treating cancer in a subject in need thereof comprising administering to the subject a therapeutically effective amount of a pharmaceutical composition comprising 1) a first therapeutic agent that is an OLIG2 modulator described herein, 2) a second therapeutic agent that is an mTOR inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (I) described herein, 2) a second therapeutic agent that is an mTOR inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (I) described herein, 2) a second therapeutic agent that is an mTOR inhibitor, and 3) at least one pharmaceutically acceptable excipient, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments is a method of treating cancer in a subject in need thereof comprising administering to the subject a therapeutically effective amount of a pharmaceutical composition comprising 1) a first therapeutic agent that is a compound of Formula (II) described herein, 2) a second therapeutic agent that is an mTOR inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (II) described herein, 2) a second therapeutic agent that is an mTOR inhibitor, and 3) at least one pharmaceutically acceptable excipient, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments of the aforementioned methods, the mTOR inhibitor is selected from rapamycin, temsirolimus, everolimus, ridaforolimus, torkinib, voxtalisib, torin 1, torin 2, omipalisib, apitolisib, gedatolisib, vistusertib, AZD8055, SF2523, CZ415, LY3023414, PI-103, KU-0063794, INK-128, OSI-027, PF-04691502, WYE-354, WYE-125132, WYE-687, BGT226, and WAY-600.

Disclosed herein is a method of treating cancer in a subject in need thereof comprising administering to the subject a therapeutically effective amount of a pharmaceutical composition comprising 1) a first therapeutic agent that is an OLIG2 modulator described herein, 2) a second therapeutic agent that is a STAT3 inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (I) described herein, 2) a second therapeutic agent that is a STAT3 inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (I) described herein, 2) a second therapeutic agent that is a STAT3 inhibitor, and 3) at least one pharmaceutically acceptable excipient, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments is a method of treating cancer in a subject in need thereof comprising administering to the subject a therapeutically effective amount of a pharmaceutical composition comprising 1) a first therapeutic agent that is a compound of Formula (II) described herein, 2) a second therapeutic agent that is a STAT3 inhibitor, and 3) at least one pharmaceutically acceptable excipient. In some embodiments is a method of treating cancer in a subject in need thereof comprising administering to the subject a therapeutically effective amount of a pharmaceutical composition comprising 1) a first therapeutic agent that is a compound of Formula (II) described herein, 2) a second therapeutic agent that is a STAT3 inhibitor, and 3) at least one pharmaceutically acceptable excipient, wherein the cancer is brain cancer, glioblastoma multiforme, medulloblastoma, astrocytomas, brain stem gliomas, meningiomas, oligodendrogliomas, melanoma, lung cancer, breast cancer, or leukemia. In some embodiments of the aforementioned methods, the STAT3 inhibitor is selected from S3I-201, stattic, niclosamide, napabucasin, cryptotanshinone, HO-3867, SH-4-54, LY5, C₁₈₈-9, LLL12, and STX-0119.

Disclosed herein is a method of treating Down's syndrome in a subject in need thereof comprising administering to the subject a therapeutically effective amount of a pharmaceutical composition comprising 1) a first therapeutic agent that is an OLIG2 modulator described herein, 2) a second therapeutic agent that is an EGFR inhibitor, and 3) at least one pharmaceutically acceptable excipient. In some embodiments is a method of treating Down's syndrome in a subject in need thereof comprising administering to the subject a therapeutically effective amount of a pharmaceutical composition comprising 1) a first therapeutic agent that is a compound of Formula (I) described herein, 2) a second therapeutic agent that is an EGFR inhibitor, and 3) at least one pharmaceutically acceptable excipient. In some embodiments is a method of treating Down's syndrome in a subject in need thereof comprising administering to the subject a therapeutically effective amount of a pharmaceutical composition comprising 1) a first therapeutic agent that is a compound of Formula (II) described herein, 2) a second therapeutic agent that is an EGFR inhibitor, and 3) at least one pharmaceutically acceptable excipient. In some embodiments of the aforementioned methods, the EGFR inhibitor is selected from gefitinib, erlotinib, lapatinib, cetuximab, panitumumab, vandetanib, necitumumab, osimertinib, tesevatinib, pelitinib, rocilitinib, and JNJ2887.

Disclosed herein is a method of treating Down's syndrome in a subject in need thereof comprising administering to the subject a therapeutically effective amount of a pharmaceutical composition comprising 1) a first therapeutic agent that is an OLIG2 modulator described herein, 2) a second therapeutic agent that is an ATM inhibitor, and 3) at least one pharmaceutically acceptable excipient. In some embodiments is a method of treating Down's syndrome in a subject in need thereof comprising administering to the subject a therapeutically effective amount of a pharmaceutical composition comprising 1) a first therapeutic agent that is a compound of Formula (I) described herein, 2) a second therapeutic agent that is an ATM inhibitor, and 3) at least one pharmaceutically acceptable excipient. In some embodiments is a method of treating Down's syndrome in a subject in need thereof comprising administering to the subject a therapeutically effective amount of a pharmaceutical composition comprising 1) a first therapeutic agent that is a compound of Formula (II) described herein, 2) a second therapeutic agent that is an ATM inhibitor, and 3) at least one pharmaceutically acceptable excipient. In some embodiments of the aforementioned methods, the ATM inhibitor is selected from KU-55933, KU-60019, wortmannin, torin 2, CP-466722, and CGK-733.

Disclosed herein is a method of treating Down's syndrome in a subject in need thereof comprising administering to the subject a therapeutically effective amount of a pharmaceutical composition comprising 1) a first therapeutic agent that is an OLIG2 modulator described herein, 2) a second therapeutic agent that is an ATR inhibitor, and 3) at least one pharmaceutically acceptable excipient. In some embodiments is a method of treating Down's syndrome in a subject in need thereof comprising administering to the subject a therapeutically effective amount of a pharmaceutical composition comprising 1) a first therapeutic agent that is a compound of Formula (I) described herein, 2) a second therapeutic agent that is an ATR inhibitor, and 3) at least one pharmaceutically acceptable excipient. In some embodiments is a method of treating Down's syndrome in a subject in need thereof comprising administering to the subject a therapeutically effective amount of a pharmaceutical composition comprising 1) a first therapeutic agent that is a compound of Formula (II) described herein, 2) a second therapeutic agent that is an ATR inhibitor, and 3) at least one pharmaceutically acceptable excipient. In some embodiments of the aforementioned methods, the ATR inhibitor is selected from dactolisib, VE-821, VE-822, ETP-46464, CGK-733, AZ-20, and AZD-6738.

Disclosed herein is a method of treating Down's syndrome in a subject in need thereof comprising administering to the subject a therapeutically effective amount of a pharmaceutical composition comprising 1) a first therapeutic agent that is an OLIG2 modulator described herein, 2) a second therapeutic agent that is a PARP inhibitor, and 3) at least one pharmaceutically acceptable excipient. In some embodiments is a method of treating Down's syndrome in a subject in need thereof comprising administering to the subject a therapeutically effective amount of a pharmaceutical composition comprising 1) a first therapeutic agent that is a compound of Formula (I) described herein, 2) a second therapeutic agent that is a PARP inhibitor, and 3) at least one pharmaceutically acceptable excipient. In some embodiments is a method of treating Down's syndrome in a subject in need thereof comprising administering to the subject a therapeutically effective amount of a pharmaceutical composition comprising 1) a first therapeutic agent that is a compound of Formula (II) described herein, 2) a second therapeutic agent that is a PARP inhibitor, and 3) at least one pharmaceutically acceptable excipient. In some embodiments of the aforementioned methods, the PARP inhibitor is selected from niraparib, iniparib, talazoparib, veliparib, oloparib, rucaparib, CEP-9722, E7106, and BGB-290.

Disclosed herein is a method of treating Down's syndrome in a subject in need thereof comprising administering to the subject a therapeutically effective amount of a pharmaceutical composition comprising 1) a first therapeutic agent that is an OLIG2 modulator described herein, 2) a second therapeutic agent that is a CDK4/6 inhibitor, and 3) at least one pharmaceutically acceptable excipient. In some embodiments is a method of treating Down's syndrome in a subject in need thereof comprising administering to the subject a therapeutically effective amount of a pharmaceutical composition comprising 1) a first therapeutic agent that is a compound of Formula (I) described herein, 2) a second therapeutic agent that is a CDK4/6 inhibitor, and 3) at least one pharmaceutically acceptable excipient. In some embodiments is a method of treating Down's syndrome in a subject in need thereof comprising administering to the subject a therapeutically effective amount of a pharmaceutical composition comprising 1) a first therapeutic agent that is a compound of Formula (II) described herein, 2) a second therapeutic agent that is a CDK4/6 inhibitor, and 3) at least one pharmaceutically acceptable excipient. In some embodiments of the aforementioned methods, the CDK4/6 inhibitor is selected from palbociclib, abemaciclib, P1446A-05, ribociclib, R547, LY2835219, alvocidib, PHA-793887, P276-00, AT7519, milciclib, SU9516, BMS-265246, JNJ-7706621, SNS-032, LDC000067, and LEE011.

Disclosed herein is a method of treating Down's syndrome in a subject in need thereof comprising administering to the subject a therapeutically effective amount of a pharmaceutical composition comprising 1) a first therapeutic agent that is an OLIG2 modulator described herein, 2) a second therapeutic agent that is a Chk1 inhibitor, and 3) at least one pharmaceutically acceptable excipient. In some embodiments is a method of treating Down's syndrome in a subject in need thereof comprising administering to the subject a therapeutically effective amount of a pharmaceutical composition comprising 1) a first therapeutic agent that is a compound of Formula (I) described herein, 2) a second therapeutic agent that is a Chk1 inhibitor, and 3) at least one pharmaceutically acceptable excipient. In some embodiments is a method of treating Down's syndrome in a subject in need thereof comprising administering to the subject a therapeutically effective amount of a pharmaceutical composition comprising 1) a first therapeutic agent that is a compound of Formula (II) described herein, 2) a second therapeutic agent that is a Chk1 inhibitor, and 3) at least one pharmaceutically acceptable excipient. In some embodiments of the aforementioned methods, the Chk1 inhibitor is selected from CHIR-124, PF-477736, MK-8776, LY2603618, and AZD7762.

Disclosed herein is a method of treating Down's syndrome in a subject in need thereof comprising administering to the subject a therapeutically effective amount of a pharmaceutical composition comprising 1) a first therapeutic agent that is an OLIG2 modulator described herein, 2) a second therapeutic agent that is a JAK2 inhibitor, and 3) at least one pharmaceutically acceptable excipient. In some embodiments is a method of treating Down's syndrome in a subject in need thereof comprising administering to the subject a therapeutically effective amount of a pharmaceutical composition comprising 1) a first therapeutic agent that is a compound of Formula (I) described herein, 2) a second therapeutic agent that is a JAK2 inhibitor, and 3) at least one pharmaceutically acceptable excipient. In some embodiments is a method of treating Down's syndrome in a subject in need thereof comprising administering to the subject a therapeutically effective amount of a pharmaceutical composition comprising 1) a first therapeutic agent that is a compound of Formula (II) described herein, 2) a second therapeutic agent that is a JAK2 inhibitor, and 3) at least one pharmaceutically acceptable excipient. In some embodiments of the aforementioned methods, the JAK2 inhibitor is selected from ruxolitnib, tofacitinib, baricitinib, filgotinib, gandotinib, lestaurtinib, momelotinib, pacritinib, upadacitinib, fedratinib, cerdulatinib, AZD1480, AZ 960, NVP-BSK805, CEP-33779, TG101209, WP1066, AG-490, GLPG0634, Go6976, LY2784544, and AT9283.

Disclosed herein is a method of treating Down's syndrome in a subject in need thereof comprising administering to the subject a therapeutically effective amount of a pharmaceutical composition comprising 1) a first therapeutic agent that is an OLIG2 modulator described herein, 2) a second therapeutic agent that is an mTOR inhibitor, and 3) at least one pharmaceutically acceptable excipient. In some embodiments is a method of treating Down's syndrome in a subject in need thereof comprising administering to the subject a therapeutically effective amount of a pharmaceutical composition comprising 1) a first therapeutic agent that is a compound of Formula (I) described herein, 2) a second therapeutic agent that is an mTOR inhibitor, and 3) at least one pharmaceutically acceptable excipient. In some embodiments is a method of treating Down's syndrome in a subject in need thereof comprising administering to the subject a therapeutically effective amount of a pharmaceutical composition comprising 1) a first therapeutic agent that is a compound of Formula (II) described herein, 2) a second therapeutic agent that is an mTOR inhibitor, and 3) at least one pharmaceutically acceptable excipient. In some embodiments of the aforementioned methods, the mTOR inhibitor is selected from rapamycin, temsirolimus, everolimus, ridaforolimus, torkinib, voxtalisib, torin 1, torin 2, omipalisib, apitolisib, gedatolisib, vistusertib, AZD8055, SF2523, CZ415, LY3023414, PI-103, KU-0063794, INK-128, OSI-027, PF-04691502, WYE-354, WYE-125132, WYE-687, BGT226, and WAY-600.

Disclosed herein is a method of treating Down's syndrome in a subject in need thereof comprising administering to the subject a therapeutically effective amount of a pharmaceutical composition comprising 1) a first therapeutic agent that is an OLIG2 modulator described herein, 2) a second therapeutic agent that is a STAT3 inhibitor, and 3) at least one pharmaceutically acceptable excipient. In some embodiments is a method of treating Down's syndrome in a subject in need thereof comprising administering to the subject a therapeutically effective amount of a pharmaceutical composition comprising 1) a first therapeutic agent that is a compound of Formula (I) described herein, 2) a second therapeutic agent that is a STAT3 inhibitor, and 3) at least one pharmaceutically acceptable excipient. In some embodiments is a method of treating Down's syndrome in a subject in need thereof comprising administering to the subject a therapeutically effective amount of a pharmaceutical composition comprising 1) a first therapeutic agent that is a compound of Formula (II) described herein, 2) a second therapeutic agent that is a STAT3 inhibitor, and 3) at least one pharmaceutically acceptable excipient. In some embodiments of the aforementioned methods, the STAT3 inhibitor is selected from S3I-201, stattic, niclosamide, napabucasin, cryptotanshinone, HO-3867, SH-4-54, LY5, C₁₈₈-9, LLL12, and STX-0119.

### Pharmaceutical Compositions and Methods of Administration

Disclosed herein, in certain embodiments, are pharmaceutical compositions comprising a first therapeutic agent that is a compound of Formula (I) or (II), a second therapeutic agent, and at least one pharmaceutically acceptable excipient. In some embodiments is a pharmaceutical composition comprising a first therapeutic agent that is a compound of Formula (I) or (II), a second therapeutic agent that is an epidermal growth factor receptor (EGFR) inhibitor, an ataxia telangiectasia mutated (ATM) kinase inhibitor, an ataxia telengiectasia and Rad3 related (ATR) kinase inhibitor, a poly ADP-ribose polymerase (PARP) inhibitor, a cyclin-dependent kinase (CDK) 4/6 inhibitor, a checkpoint kinase 1 (Chk1) inhibitor, a signal transducer and activator of transcription 3 (STAT3) inhibitor, a mechanistic target of rapamycin (mTOR) inhibitor, or a Janus Kinase 2 (JAK2) inhibitor, and at least one pharmaceutically acceptable excipient. In some embodiments is a pharmaceutical composition comprising a first therapeutic agent that is a compound of Formula (I) or (II), a second therapeutic agent that is an epidermal growth factor receptor (EGFR) inhibitor, and at least one pharmaceutically acceptable excipient. In some embodiments is a pharmaceutical composition comprising a first therapeutic agent that is a compound of Formula (I) or (II), a second therapeutic agent that is an ataxia telangiectasia mutated (ATM) kinase inhibitor, and at least one pharmaceutically acceptable excipient. In some embodiments is a pharmaceutical composition comprising a first therapeutic agent that is a compound of Formula (I) or (II), a second therapeutic agent that is an ataxia telengiectasia and Rad3 related (ATR) kinase inhibitor, and at least one pharmaceutically acceptable excipient. In some embodiments is a pharmaceutical composition comprising a first therapeutic agent that is a compound of Formula (I) or (II), a second therapeutic agent that is a poly ADP-ribose polymerase (PARP) inhibitor, and at least one pharmaceutically acceptable excipient. In some embodiments is a pharmaceutical composition comprising a first therapeutic agent that is a compound of Formula (I) or (II), a second therapeutic agent that is a cyclin-dependent kinase (CDK) 4/6 inhibitor, and at least one pharmaceutically acceptable excipient. In some embodiments is a pharmaceutical composition comprising a first therapeutic agent that is a compound of Formula (I) or (II), a second therapeutic agent that is a checkpoint kinase 1 (Chk1) inhibitor, and at least one pharmaceutically acceptable excipient. In some embodiments is a pharmaceutical composition comprising a first therapeutic agent that is a compound of Formula (I) or (II), a second therapeutic agent that is a signal transducer and activator of transcription 3 (STAT3) inhibitor, and at least one pharmaceutically acceptable excipient. In some embodiments is a pharmaceutical composition comprising a first therapeutic agent that is a compound of Formula (I) or (II), a second therapeutic agent that is a mechanistic target of rapamycin (mTOR) inhibitor, and at least one pharmaceutically acceptable excipient. In some embodiments is a pharmaceutical composition comprising a first therapeutic agent that is a compound of Formula (I) or (II), a second therapeutic agent that is a Janus Kinase 2 (JAK2) inhibitor, and at least one pharmaceutically acceptable excipient.

The pharmaceutical compositions described herein are formulated using one or more physiologically acceptable excipients which facilitate processing of the active compounds into preparations which can be used pharmaceutically. Proper formulation is dependent upon the route of administration chosen. A summary of pharmaceutical compositions described herein is found, for example, in Remington: The Science and Practice of Pharmacy, Twentysecond Ed (Pharmaceutical Press, 2012); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some embodiments, the first therapeutic agent that is a compound of Formula (I) or (II) and second therapeutic agent are administered concurrently (simultaneously, essentially simultaneously, or within the same treatment protocol) or sequentially, depending upon the nature of the diseases, the condition of the patient, and the actual choice of compounds used.

In certain embodiments, the determination of the order of administration, and the number of repetitions of administration of each therapeutic agent during a treatment protocol, is based upon evaluation of the disease being treated and the condition of the patient.

In certain embodiments, the first therapeutic agent that is a compound of Formula (I) or (II) and second therapeutic agent are part of the same composition (fixed combination). In some embodiments, the compound of Formula (I) or (II) and the second therapeutic agent are administered as different compositions (non-fixed combinations). In another embodiment, the compound of Formula (I) or (II) is administered prior to the second therapeutic agent. In some embodiments, the second therapeutic agent is administered prior to the compound of Formula (I) or (II). As many of the disorders for which the compounds and compositions of the invention are useful in treating are chronic disorders, in one embodiment combination therapy involves alternating between administering a compound of Formula (I) or (II) and a second therapeutic agent, e.g., to minimize the toxicity associated with a particular drug. The duration of administration of each therapeutic agent can be one day, one week, one month, three months, six months, or a year.

In some embodiments, the initial administration of a compound of Formula (I) or (II) and the second therapeutic agent are via any route practical, such as, for example, an intravenous injection, a bolus injection, infusion over 5 minutes to about 5 hours, a pill, a capsule, transdermal patch, buccal delivery, and the like, or combination thereof.

The compound of Formula (I) or (II) and the second therapeutic agent should be administered as soon as is practicable after the onset of a disorder is detected or suspected, and for a length of time necessary for the treatment of the disease, such as, for example, from about 1 month to about 3 months, or continuously throughout the individual's life. The length of treatment can vary for each subject, and the length can be determined using the known criteria. In some embodiments, the compound of Formula (I) or (II) and the second therapeutic agent are administered for at least 2 weeks, between about 1 month to about 5 years, or from about 1 month to about 3 years. In some embodiments, the compound of Formula (I) or (II) and the second therapeutic agent are administered throughout the individual's life.

Therapeutically effective amounts will depend on the severity and course of the disorder, previous therapy, the patient's health status, weight, and response to the drugs, and the judgment of the treating physician. Prophylactically effective amounts depend on the patient's state of health, weight, the severity and course of the disease, previous therapy, response to the drugs, and the judgment of the treating physician.

In some embodiments, the compound of Formula (I) or (II) and the second therapeutic agent are administered to the patient on a regular basis, e.g., three times a day, two times a day, once a day, every other day, or every 3 days. In other embodiments, the compound of Formula (I) or (II) and the second therapeutic agent are administered to the patient on an intermittent basis, e.g., twice a day followed by once a day followed by three times a day; or the first two days of every week; or the first, second and third day of a week. In some embodiments, intermittent dosing is as effective as regular dosing. In further or alternative embodiments, the compound of Formula (I) or (II) and the second therapeutic agent are administered only when the patient exhibits a particular symptom, e.g., the onset of pain, or the onset of a fever, or the onset of an inflammation, or the onset of a skin disorder. Dosing schedules of each compound may depend on the other or may be independent of the other.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance regimen is administered if necessary. Subsequently, the dosage or the frequency of administration, or both, of the compound of Formula (I) or (II) and the second therapeutic agent can be reduced, as a function of the symptoms, to a level at which the individual's improved condition is retained. Individuals can, however, require intermittent treatment on a long-term basis upon any recurrence of symptoms.

The amount of the compound of Formula (I) or (II) and the second therapeutic agent are will vary depending upon factors such as the particular compound, disorder and its severity, the identity (e.g., weight) of the subject or host in need of treatment, and is determined according to the particular circumstances surrounding the case, including, e.g., the specific agents being administered, the routes of administration, and the subject or host being treated. In general, however, doses employed for adult human treatment will typically be in the range of 0.02-5000 mg per day.

In some embodiments, the effective dose of the compound of Formula (I) or (II) is about 1 mg to about 1500 mg, about 1 mg to about 1400 mg, about 1 mg to about 1300 mg, about 1 mg to about 1200 mg, about 1 mg to about 1100 mg, about 1 mg to about 1000 mg, 1 mg to about 900 mg, about 1 mg to about 800 mg, about 1 mg to about 700 mg. In some embodiments, the effective dose of the compound of Formula (I) or (II) is about 1 mg to about 600 mg, about 1 mg to about 500 mg, about 1 mg to about 400 mg, about 1 mg to about 300 mg, about 1 mg to about 200 mg, about 1 mg to about 100 mg, about 1 mg to about 90 mg, about 1 mg to about 80 mg, about 1 mg to about 70 mg, about 1 mg to about 60 mg, about 1 mg to about 50 mg, about 1 mg to about 40 mg, about 1 mg to about 30 mg, about 1 mg to about 20 mg, about 1 mg to about 10 mg, or about 1 mg to about 5 mg.

In some embodiments, the effective dose of the compound of Formula (I) or (II) is about 10 mg to about 1500 mg, about 20 mg to about 1500 mg, about 30 mg to about 1500 mg, about 40 mg to about 1500 mg, about 50 mg to about 1500 mg, about 60 mg to about 1500 mg, about 70 mg to about 1500 mg, about 80 mg to about 1500 mg, about 90 mg to about 1500 mg, about 100 mg to about 1500 mg, about 200 mg to about 1500 mg, about 300 mg to about 1500 mg, about 400 mg to about 1500 mg, about 500 mg to about 1500 mg, about 600 mg to about 1500 mg, about 700 mg to about 1500 mg, about 800 mg to about 1500 mg, about 900 mg to about 1500 mg, about 1000 mg to about 1500 mg, about 1100 mg to about 1500 mg, about 1200 mg to about 1500 mg, about 1300 mg to about 1500 mg, or about 1400 mg to about 1500 mg. In some embodiments, the effective dose of the compound of Formula (I) or (II) is about 10 mg to about 450 mg, about 10 mg to about 50 mg, about 50 mg to about 100 mg, about 100 mg to about 200 mg, about 200 mg to about 300 mg, or about 300 mg to about 450 mg. In some embodiments, the effective dose of the compound of Formula (I) or (II) is about 1 mg to about 100 mg, about 1 mg to about 5 mg, about 5 mg to about 10 mg, about 10 mg to about 25 mg, about 25 mg to about 50 mg, about 50 mg to about 75 mg, or about 75 mg to about 100 mg. In some embodiments, the effective dose of the compound of Formula (I) or (II) is about 10 mg to about 250 mg, about 10 mg to about 50 mg, about 50 mg to about 100 mg, about 100 mg to about 150 mg, about 150 mg to about 200 mg, or about 200 mg to about 250 mg. In some embodiments, the effective dose of the compound of Formula (I) or (II) is about 500 mg to about 1500 mg, about 500 mg to about 600 mg, about 600 mg to about 700 mg, about 700 mg to about 800 mg, about 800 mg to about 900 mg, about 900 mg to about 1000 mg, about 1000 mg to about 1200 mg, or about 1200 mg to about 1500 mg.

In some embodiments, the effective dose of the second therapeutic agent is about 1 mg to about 1000 mg. In some embodiments, the effective dose of the second therapeutic agent is about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 20.5 mg, about 21 mg, about 21.5 mg, about 22 mg, about 22.5 mg, about 23 mg, about 23.5 mg, about 24 mg, about 24.5 mg, about 25 mg, about 25.5 mg, about 26 mg, about 26.5 mg, about 27 mg, about 27.5 mg, about 28 mg, about 28.5 mg, about 29 mg, about 29.5 mg, about 30 mg, about 30.5 mg, about 31 mg, about 31.5 mg, about 32 mg, about 32.5 mg, about 33 mg, about 33.5 mg, about 34 mg, about 34.5 mg, about 35 mg, about 35.5 mg, about 36 mg, about 36.5 mg, about 37 mg, about 37.5 mg, about 38 mg, about 38.5 mg, about 39 mg, about 39.5 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 750 mg, about 800 mg, about 850 mg, about 900 mg, or about 950 mg, about 1000 mg. In certain embodiments, any two of the doses in this paragraph may be combined to form a range of dosages included within the disclosure, e.g., the effective dose of the second therapeutic agent is from about 2 mg to about 100 mg, from about 10 mg to about 150 mg, from about 50 mg to about 200 mg, or from about 100 mg to about 300 mg.

It is understood that a medical professional will determine the dosage regimen in accordance with a variety of factors. These factors include the age, weight, sex, diet, and medical condition of the subject.

In some embodiments, a pharmaceutical composition, refers to a mixture of a compound of Formula (I) or (II) and a second therapeutic agent with other chemical components, such as carriers, stabilizers, diluents, dispersing agents, suspending agents, thickening agents, and/or excipients. In some embodiments, a pharmaceutical composition refers to a compound of Formula (I) or (II) with other chemical components, such as carriers, stabilizers, diluents, dispersing agents, suspending agents, thickening agents, and/or excipients. In some embodiments, a pharmaceutical composition refers to a second therapeutic agent with other chemical components, such as carriers, stabilizers, diluents, dispersing agents, suspending agents, thickening agents, and/or excipients.

Pharmaceutical compositions are optionally manufactured in a conventional manner, such as, by way of example only, by means of conventional mixing, dissolving, granulating, dragee-making, levigating, emulsifying, encapsulating, entrapping or compression processes.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite, and ammonium sulfate.

The pharmaceutical formulations described herein are administered by any suitable administration route, including but not limited to, oral, parenteral (e.g., intravenous, subcutaneous, intramuscular), intranasal, buccal, topical, rectal, or transdermal administration routes.

The pharmaceutical compositions described herein are formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the compositions are formulated into capsules. In some embodiments, the compositions are formulated into solutions (for example, for IV administration).

The pharmaceutical compositions described herein are formulated into unit dosage forms suitable for single administration of precise dosages. In unit dosage form, the formulation is divided into unit doses containing appropriate quantities of one or both compounds. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The pharmaceutical solid dosage forms described herein optionally include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating is provided around the compositions. In some embodiments, the compositions are formulated into particles (for example for administration by capsule) and some or all of the particles are coated. In some embodiments, the compositions are formulated into particles (for example for administration by capsule) and some or all of the particles are microencapsulated. In some embodiments, the compositions are formulated into particles (for example for administration by capsule) and some or all of the particles are not microencapsulated and are uncoated.

In certain embodiments, compositions provided herein may also include one or more preservatives to inhibit microbial activity. Suitable preservatives include mercury-containing substances such as merfen and thiomerosal; stabilized chlorine dioxide; and quaternary ammonium compounds such as benzalkonium chloride, cetyltrimethylammonium bromide and cetylpyridinium chloride.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans, and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol, and isopropyl myristate.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

The term “disintegrate” includes both the dissolution and dispersion of the dosage form when contacted with gastrointestinal fluid. “Disintegration agents or disintegrants” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids. Combinations of one or more erosion facilitator with one or more diffusion facilitator can also be used in the present compositions.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce or inhibit adhesion or friction of materials. Exemplary lubricants include, e.g., stearic acid, calcium hydroxide, talc, sodium stearyl fumarate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Steady state,” as used herein, is when the amount of drug administered is equal to the amount of drug eliminated within one dosing interval resulting in a plateau or constant plasma drug exposure.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

It should be appreciated that there is considerable overlap between additives used in the solid dosage forms described herein. Thus, the above-listed additives should be taken as merely exemplary, and not limiting, of the types of additives that can be included in solid dosage forms described herein. The amounts of such additives can be readily determined by one skilled in the art, according to the particular properties desired.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

Compressed tablets are solid dosage forms prepared by compacting the bulk blend of the formulations described above. In various embodiments, compressed tablets which are designed to dissolve in the mouth will include one or more flavoring agents. In other embodiments, the compressed tablets will include a film surrounding the final compressed tablet. In some embodiments, the film coating can provide a delayed release of the compound of Formula (I) or (II) in combination with or separately from a second therapeutic agent, from the formulation. In other embodiments, the film coating aids in patient compliance (e.g., Opadry® coatings or sugar coating). Film coatings including Opadry® typically range from about 1% to about 3% of the tablet weight. In other embodiments, the compressed tablets include one or more excipients.

A capsule may be prepared, for example, by placing the bulk blend of the formulation of the compounds described herein, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles of the compounds described herein and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In another aspect, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with the compounds described herein, which sufficiently isolate the compounds from other non-compatible excipients. In some embodiments, materials compatible with compounds of Formula (I) or (II) and the second therapeutic agent are those that delay the release of the compounds of Formula (I) or (II) and the second therapeutic agent, in vivo.

Exemplary microencapsulation materials useful for delaying the release of the formulations including compounds described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

Microencapsulated compounds of the compounds described herein may be formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

In one embodiment, the particles of the compounds described herein are microencapsulated prior to being formulated into one of the above forms. In still another embodiment, some or most of the particles are coated prior to being further formulated by using standard coating procedures, such as those described in Remington's PharmaceuticalSciences, 20th Edition (2000).

In still other embodiments, effervescent powders are also prepared in accordance with the present disclosure. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the compositions described herein are added to water, the acids and the base react to liberate carbon dioxide gas, thereby causing “effervescence.” Examples of effervescent salts include, e.g., the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the methods and compositions described herein to achieve delivery to the lower gastrointestinal tract. In some embodiments the polymers described herein are anionic carboxylic polymers. In other embodiments, the polymers and compatible mixtures thereof, and some of their properties, include, but are not limited to:

Shellac, also called purified lac, a refined product obtained from the resinous secretion of an insect. This coating dissolves in media of pH>7;

Acrylic polymers. The performance of acrylic polymers (primarily their solubility in biological fluids) can vary based on the degree and type of substitution. Examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;

Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP psuedolatex with particles<1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions; Poly Vinyl Acetate Phthalate (PVAP). PVAP dissolves in pH>5, and it is much less permeable to water vapor and gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., carnuba wax or PEG) may be added to the coatings besides plasticizers to solubilize or disperse the coating material, and to improve coating performance and the coated product.

In other embodiments, the formulations described herein, which include the compounds described herein, are delivered using a pulsatile dosage form. A pulsatile dosage form is capable of providing one or more immediate release pulses at predetermined time points after a controlled lag time or at specific sites. Many other types of controlled release systems known to those of ordinary skill in the art and are suitable for use with the formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

In some embodiments, pharmaceutical formulations are provided that include particles of the compounds described herein and at least one dispersing agent or suspending agent for oral administration to a subject. The formulations may be a powder and/or granules for suspension, and upon admixture with water, a substantially uniform suspension is obtained.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition the liquid dosage forms may include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

### EXAMPLES

These examples are provided for illustrative purposes only and not to limit the scope of the claims provided herein. The starting materials and reagents used for the synthesis of the compounds described herein may be synthesized or can be obtained from commercial sources, such as, but not limited to, Sigma-Aldrich, Acros Organics, Fluka, and Fischer Scientific.

### Example 1: Synergy of Cytotoxicity of 1-(3,4-Dichlorophenyl)-3-(4-(3-(dimethylamino)propylamino)-6-methylpyrimidin-2-yl)urea in Combination with an EGFR Inhibitor in Glioblastoma Cells

GBM8 or GBM4 cells were seeded at a density of 3×10⁴c/ml in a 96 well plate at 100 ul per well. The perimeter wells were not used and contained media only. Cells were cultured in StemCell NeuroCult NS-A Basal Medium containing NeuroCult NS-A Proliferation Supplement, 20 ng/ml rhEGF, 10 ng/ml bFGF, and 0.0002% Heparin.

Cells were seeded in the presence of olig2 inhibitor, 1-(3,4-dichlorophenyl)-3-(4-(3-(dimethylamino)propylamino)-6-methylpyrimidin-2-yl)urea, at increasing concentrations horizontally (i.e. row B had 0 uM the olig2 inhibitor thru to row G having the highest concentration). The final DMSO concentration in the assay plate after the addition of the olig2 inhibitor was ˜0.5%.

The next day, at approximately 24 hours, an EGFR inhibitor was added at increasing concentrations vertically (i.e. Column 2 had 0 uM the EGFR inhibitor thru to column 10 having the highest concentration). Column 11 contained cells with DMSO only. The final DMSO concentration in the assay plate after addition of the EGFR inhibitor is ˜1%.

Cell viabilities were measured 72 hours after the addition of drug B by Cell-Titer Glo (Promega) luminescence assay on the Clariostar. Data analysis was done in Graphpad Prism software. Combination studies with 1-(3,4-dichlorophenyl)-3-(4-(3-(dimethylamino)propylamino)-6-methylpyrimidin-2-yl)urea and an EGFRi result in combination indices<1 indicating synergy for cytotoxic effects (Table 1).

TABLE 1

| EGFR inhibitor | GBM8 CI |
|---|---|
| Pelitinib | 0.83 |
| Rocilitinib | 0.70 |
| JNJ2887 | 0.53 |
| XL-647 | 0.75 |

### Example 2: Synergy of Cytotoxicity of 1-(3,4-Dichlorophenyl)-3-(4-(3-(dimethylamino)propylamino)-6-methylpyrimidin-2-yl)urea in Combination with an mTOR Inhibitor in Glioblastoma Cells

Glioblastoma cancer cell lines NN01, NX03_CA, NX18_25, NN27 and control lines NP01 and NHNP were dissociated with Accutase cell detachment solution (#10210-214, VWR, Radnor, PA). 1000 cells per well were plated in an ultra-low attachment 384-well plate (#3830, Corning, Tewksbury, MA) and incubated at 37° C. in neurobasal media (#21103-049, Thermofisher, San Diego, CA) supplemented with 2 mM L-Glutamax, 1×B27 w/o vitamin A (#35050-061, #12587-010, Thermofisher, San Diego, CA), 100 U/ml Penicillin Streptomycin (#30-002-CI, Corning, Tewksbury, MA), 32 U/mL Heparin (#H3149-100KU, Sigma, St. Louis, MO), 2 μL/mL NSF-1 (#CC-4323, Lonza Walkersville Inc, Basel, CH), 20 ng/mL EGF and 20 ng/mL FGF (#AF-100-15, #, 100-18B, Peprotech, Rocky Hill, NJ). Once the neurospheres reached a sphere size of 200 μm, variable concentrations of the test compound, 1-(3,4-dichlorophenyl)-3-(4-(3-(dimethylamino)propylamino)-6-methylpyrimidin-2-yl)urea, were added and mTOR inhibitors were also added to yield a final concentration of 10 nM. The assay media had a final DMSO concentration of 0.1%. After another 72 hours, cell viability was determined using the Cell TiterGlo kit (#G7570, Promega, Madison. WI) following the manufacturer's instructions on a Tecan M200 Pro Plate Reader (Tecan, Mannedorf, CH). Percent viable cells were normalized against vehicle control (DMSO set as 100%). The percent inhibition of cell viability is shown in Table 2.

TABLE 2

| | Test | | | |
|---|---|---|---|---|
| | compound | | | |
| | @ 3 μM | Everolimus | Rapamycin | Combined |
| NX18 cells- | 30% | 45% | Not tested | 60% |
| Everolimus | | | | |
| NX18 cells- | 30% | Not tested | 41% | 61% |
| Rapamycin | | | | |

### Example 3: Phase II Clinical Trial of a Compound of Formula (I) or (II) in Combination with a Olaparib in Patients with Recurrent Rb Positive Glioblastoma

The purpose of this phase II trial is to determine the efficacy of a combination treatment of a compound of Formula (I) or (II) and Olaparib (as measured by progression free survival at 6 months) in patients with recurrent glioblastoma multiforme or gliosarcoma who are Rb positive. A total of 30 patients will be treated; 15 will undergo a planned surgical resection and receive drug for 7 days prior to surgery, followed by drug after recovery from surgery, and the other 15 patients will receive drug without a planned surgical procedure.

Patients: Eligible subjects will be men and women 18 years and older

Criteria:

Inclusion Criteria:
Patients with radiographically proven recurrent, intracranial Glioblastoma multiforme or Gliosarcoma will be eligible for this protocol. Patients must have documentation of Rb positive disease.
All patients must sign an informed consent indicating that they are aware of the investigational nature of this study. Patients must have signed an authorization for the release of their protected health information. Patients must be registered prior to treatment with study drug. Treatment must take place within 7 days of registration; if treatment is delayed more than 7 days, the laboratory tests for eligibility and history and physical exam must be repeated.
Patients must have had prior external beam radiation and temozolomide chemotherapy; there is no limit to the number of prior chemotherapies used; patients may be treated in their first, second or third relapse
Patients must be >18 years old, and with a life expectancy>8 weeks.
Patients must have a Karnofsky Performance Status of >60.
At the time of registration: Patients must have recovered from the toxic effects of prior therapy: >28 days from any investigational agent [NOTE: off-label use of FDA approved agents are not considered investigational for the purposes of this protocol], >28 days from prior cytotoxic therapy, >42 days from nitrosoureas, >28 days from bevacizumab, and >7 days for non-cytotoxic agents, e.g., interferon, tamoxifen, thalidomide, cis-retinoic acid, and erlotinib, for example. Any questions related to the definition of non-cytotoxic agents should be directed to the Study Chair.
Patients must have adequate bone marrow function (WBC>3,000/μl, ANC>1,500/mm³, platelet count of >100,000/mm³, and hemoglobin>10 gm/dl), adequate liver function (SGOT and bilirubin<2 times ULN), and adequate renal function (creatinine<1.5 mg/dL) before starting therapy. A pre-study EKG is required for all patients, and patients must have a normal QT interval. These tests must be performed within 14 days prior to registration. Eligibility level for hemoglobin may be reached by transfusion.
Patients must have shown unequivocal radiographic evidence for tumor progression by MRI scan. A scan should be performed within 14 days prior to registration and on a steroid dose that has been stable for at least 7 days. If the steroid dose is increased between the date of imaging and registration a new baseline MRI is required. The same type of scan, i.e., MRI must be used throughout the period of protocol treatment for tumor measurement. Patients unable to undergo MR imaging will not be eligible.
Patients having undergone recent resection of recurrent or progressive tumor will be eligible as long as all of the following conditions apply:
   They have recovered from the effects of surgery.
   Residual disease following resection of recurrent intracranial Glioblastoma Multiforme or Gliosarcoma is not mandated for eligibility into the study. To best assess the extent of residual disease post-operatively, an MRI should be done no later than 96 hours in the immediate post-operative period or at least 4 weeks post-operatively, within 14 days prior to registration. If the 96-hour scan is more than 14 days before registration, the scan needs to be repeated. If the steroid dose is increased between the date of imaging and registration, a new baseline MRI is required on a stable steroid dosage for at least 7 days.
Patients must have failed prior radiation therapy and temozolomide and must have an interval of greater than or equal to 42 days from the completion of radiation therapy to study entry.
Patients with prior therapy that included interstitial brachytherapy, stereotactic radiosurgery, or Gliadel wafers must have confirmation of true progressive disease rather than radiation necrosis based upon PET scanning, MR spectroscopy or surgical documentation of disease.
A subset of 15 patients will be enrolled prior to a planned, indicated surgical resection. Patients can be enrolled pre-operatively only if they are surgical candidates, do not have evidence of an acute intracranial hemorrhage and are able to start protocol treatment in a window of 7 days before surgery.
Male and female patients with reproductive potential must use an approved contraceptive method, if appropriate (for example, intrauterine device [IUD], birth control pills, or barrier device) during and for 3 months after discontinuation of study treatment. Women of childbearing potential must have a negative beta-HCG pregnancy test documented within 14 days prior to registration.
Blocks or slides of tumor tissue from a previous surgery must be available to do IHC Rb staining. Patients with negative tumors (Rb negative) will be excluded from the study.

Exclusion Criteria:
Patients must not have any significant medical illnesses that in the investigator's opinion cannot be adequately controlled with appropriate therapy or would compromise the patient's ability to tolerate this therapy.
Patients with a history of any other cancer (except non-melanoma skin cancer or carcinoma in-situ of the cervix), unless in complete remission and off of all therapy for that disease for a minimum of 3 years are ineligible.
Patients must not have an active infection or serious intercurrent medical illness. Patients with a history of acute intracranial hemorrhage will also be excluded.
Patients must not be pregnant/breast feeding and must agree to practice adequate contraception.
Patients must not have any disease that will obscure toxicity or dangerously alter drug metabolism.
Because of the potential for drug interactions, patients on enzyme-inducing anti-epileptic drugs or other drugs that cause CYP3A enzyme induction or inhibition will not be eligible unless they are off therapy for at least 14 days
Patients with congenital or other reasons for prolongation of the QT interval on EKG will be excluded.

Study Design: A total of 30 patients with recurrent Glioblastoma or Gliosarcoma will be treated with a combination of a compound of Formula (I) or (II) and Olaparib daily for 21 consecutive days followed by a 7 day break off therapy (cycle length is 28 days). Of these 30 patients, 15 will receive drug for 7 days prior to an indicated, intended surgical resection for progression, and will then resume drug at the same dose after recovery from surgery. Treatment will be repeated every 28 days, and in the absence of disease progression patients may receive treatment for 12 cycles. At that time patients will be given the option to continue on study past 12 cycles, up to a maximum of 24 cycles.

Following registration, available blocks or slides from a previous surgery must be submitted for diagnosis review (confirmation of Glioblastoma multiforme or Gliosarcoma) and Rb status determination. Only patients with Rb positive tumors can be treated, and Rb tumor status must be known prior to any treatment. Additional tissue from previous surgeries will also be obtained to evaluate molecular abnormalities in the tumor. These studies will be done retrospectively and are not required to be performed prior to registration.

Monitoring will include a clinical and neurological exam before the beginning of each cycle (every 4 weeks). Complete blood counts with differential will be examined on days 1 and 15 of each cycle. Liver and renal function will be performed every 4 weeks. Toxicity and dose modifications will be based on the NCI CTCAE Version 4. Disease status will be assessed clinically each cycle (every 4 weeks) and radiographically after each second cycle (every 8 weeks).

Primary Outcome Measures:
Efficacy as determined by progression free survival [Time Frame: 1-2 years] [Designated as safety issue: No]
Determine the efficacy of the compound of Formula (I) or (11)/Olaparib combination in patients with recurrent glioblastoma multiforme or gliosarcoma who are Rb positive, as measured by progression free survival at 6 months. A total of 30 patients will be treated; 15 who will undergo a planned, intended surgical resection will receive drug for 7 days prior to surgery, followed by drug after recovery from surgery, and 15 patients who receive drug without a planned surgical procedure

Secondary Outcome Measures:
Number of Participants with Adverse Events as a Measure of Safety and Tolerability [Time Frame: 1-2 years] [Designated as safety issue: Yes]

### Example 4: Phase II Clinical Trial of the Safety and Efficacy of a Compound of Formula (I) or (II) in Combination with Palbociclib in Adults with Recurrent or Refractory Medulloblastoma

The purpose of this phase II trial is to how well a combination of a compound of Formula (I) or (II) and Palbociclib works in treating adult patients with recurrent or refractory medulloblastoma.

Patients: Eligible subjects will be men and women 22 years and older.

Criteria:

Inclusion Criteria:
Patients with a histologically confirmed diagnosis of medulloblastoma (including posterior fossa PNET) that is recurrent, progressive, or refractory to standard therapy and for which there is no known curative therapy are eligible; there must be evidence of residual measurable disease or lesion in pre-study MRI as described in section; patients with spinal disease that is measurable will be eligible
The diagnosis should be confirmed at the treating institution and tissue (either from the diagnosis or relapse or preferably from both time points) must be available for biological studies
Patients with neurological deficits should have deficits that are stable for a minimum of 1 week prior to registration; this is to be documented in the database
Eastern Cooperative Oncology Group (ECOG) performance status 0-2
No other myelosuppressive chemotherapy or immunotherapy within 4 weeks prior to study entry (6 weeks if prior nitrosourea)
Decadron dose should also be stable or decreasing for at least 1 week (7 days) prior to starting therapy
Radiation therapy (XRT)>=3 months prior to study entry for craniospinal irradiation (>=23 Gy); >=8 weeks for local irradiation to primary tumor; >=2 weeks prior to study entry for focal irradiation for symptomatic metastatic sites
Off all colony stimulating factors>=1 week prior to study entry (GCSF, GM CSF, erythropoietin)
Absolute neutrophil count (ANC)>=1000/μL
Platelet count>=50,000/uL (transfusion independent)
Hemoglobin>=8.0 gm/dL (may receive RBC transfusions)
Creatinine clearance or radio-isotope GFR>=70 ml/min/1.73 m2 or
A serum creatinine=<2.0 mg/dL
Total bilirubin=<1.5× upper limit of normal (ULN) for age
Serum glutamic pyruvic transaminase (SGPT) (alanine aminotransferase [ALT])=<2.5× institutional ULN
Serum glutamic-oxalacetic transaminase (SGOT) (aspartate aminotransferase [AST])=<2.5 times institutional ULN
Serum albumin>=2.5 g/dL
Patient must have recovered from the significant acute toxicities of all prior therapy before entering this study and meet all other eligibility criteria
Pregnancy should be avoided for 12 months after the last dose for females of child-bearing potential; female patients of childbearing potential must not be pregnant or breast-feeding; female patients of childbearing potential must have a negative serum or urine pregnancy test within 24 hours prior to beginning treatment
Women of childbearing potential are required to use 2 forms of acceptable contraception, including one barrier method during participation in the study and for the 12 months following the last dose; for medical or personal reasons, 100% commitment to abstinence is considered an acceptable form of birth control. All patients should receive contraceptive counseling either by the investigator, or by an OB/gynecologist or other physician who is qualified in this area of expertise
Signed informed consent according to institutional guidelines must be obtained

Exclusion Criteria:
Patients with any clinically significant unrelated systemic illness (serious infections or significant cardiac, pulmonary, hepatic or other organ dysfunction), that would compromise the patient's ability to tolerate protocol therapy or would likely interfere with the study procedures or results
Patients receiving any other anticancer or investigational drug therapy
Patients with inability to return for follow-up visits or obtain follow-up studies required to assess toxicity to therapy
Life expectancy<12 weeks as determined by treating physician
Inability to swallow capsules
Malabsorption syndrome or other condition that would interfere with enteral absorption
History of congestive heart failure
History of ventricular arrhythmia requiring medication
Uncontrolled hypocalcemia, hypomagnesemia, hyponatremia or hypokalemia defined as less than the lower limit of normal for the institution despite adequate electrolyte supplementation
Congenital long QT syndrome

Study Design: Patients receive a compound of Formula (I) or (II) and Palbociclib PO once daily on days 1-28. Treatment repeats every 28 days for up to 26 courses in the absence of disease progression or unacceptable toxicity.

Primary Outcomes:
Objective response rates (PR and CR) graded using RECIST criteria [Time Frame: Up to 12 months] [Designated as safety issue: No]
Ninety-five percent confidence interval estimates of the true, unknown objective response rate will be constructed for each of the three strata. The proportions of patients with confirmed complete responses, partial responses and stable disease will be reported descriptively for each of the three strata. Cumulative incidence functions of time to objective response will also be provided.

Secondary Outcomes:
Duration of sustained objective response [Time Frame: From the initial scan documenting complete or partial response that was subsequently confirmed until the earlier of documented progression or death on study, assessed up to 12 months] [Designated as safety issue: No]
Progression-free survival [Time Frame: From the date of initial treatment with a compound of Formula (I) or (II) until the earliest of progression or death on study, assessed up to 12 months] [Designated as safety issue: No]
Medical costs during the first 6 months after transplantation
Patient and graft survival

### Example 5: Phase I/II Clinical Trial of the Safety, Tolerability, and Anti-tumor Efficacy of a Compound of Formula (I) or (II) in Combination with VE821 in the Treatment of Recurrent Malignant Astrocytomas

This is a single-center, open-label, non-randomized, Phase I/IIa study to investigate the safety, tolerability, and antitumor efficacy of a combination of a compound of Formula (I) or (II) and VE821 in patients with recurrent malignant astrocytomas (glioblastoma, gliosarcoma, anaplastic astrocytoma, anaplastic oligodendroglioma, anaplastic oligoastrocytoma, and anaplastic ependymoma). Patients will be treated for up to 5 cycles. A treatment cycle is defined as 28 days+7 days rest (28+7 days during cycle 1 to 4, and 28 days during cycle 5). The following cycle will not be started until the treatment continuation criteria are fulfilled. Concomitant supportive therapies will be allowed.

Patients: Eligible subjects will be men and women ages 18 and older

Criteria:

Inclusion Criteria:
Be informed of the nature of the study and have provided written informed consent
At least 18 years of age
ECOG performance of 0, 1, or 2, or KPS (Karnofsky performance status)>60.
Pathological verification of a WHO grade 4 astrocytoma (glioblastoma or gliosarcoma), or WHO Grade 3 anaplastic astrocytoma, anaplastic oligodendroglioma, anaplastic oligoastrocytoma, or anaplastic ependymoma.
Documented recurrent glioblastoma, gliosarcoma, anaplastic astrocytoma, anaplastic oligodendroglioma, anaplastic oligoastrocytoma, or anaplastic ependymoma after at least one failed treatment of chemotherapy and radiation
Expected survival of at least 3 months
At least 2-weeks from cytoreductive surgery, if performed, 4-weeks from bevacizumab or other chemotherapy (6-weeks if prior chemotherapy was nitrosourea) and 12-weeks from completion of radiotherapy.
Ability to undergo MRI scanning without and with imaging dye on a periodic basis as defined in the protocol
At least seven (7) days off of medications with induce CYP2C9 and CYP3A4 before administration of the first dose of a compound of Formula (I) or (II)
Preserved major organ functions, i.e.: Blood leukocyte count≥3.0×109/L Blood absolute neutrophil count≥1.5×109/L Blood platelet count≥100×109/L Blood hemoglobin≥100 g/L (transfusions are allowed) Plasma total bilirubin level≤1.5 times the upper institutional limit (ULN) of the ∥normal∥ (i.e. reference) range Plasma AST (aspartate aminotransferase) or ALT≤2.5 times upper institutional limit (ULN) of the ∥normal∥ range Plasma creatinine≤1.5 times upper institutional limit (ULN) of the ∥normal∥ range 12-lead ECG with normal tracings; or changes that are not clinically significant and do not require medical intervention, and QTc<500 ms At least seven (7) days off of medications which inhibit or induce CYP2C9 or CYP3A4 before first study treatment day.

Exclusion Criteria:
Ongoing infection or other major recent or ongoing disease that, according to the Investigator, poses an unacceptable risk to the patient
Grade 3 or higher constipation within the past 28 days or grade 2 constipation within the past 14 days before randomization. (Patients with grade 2 constipation within the past 14 days could be re-screened if constipation decreases to ≤grade 1 with optimal management of constipation.)
Coexisting uncontrolled medical condition, including, but not limited to, active cardiac disease and significant dementia
Hepatitis B or Hepatitis C, or HIV infection requiring anti-retroviral therapy
Active malignancy other than basal cell skin cancer
Other active malignancy during the previous 3 years
Major surgical procedure within 4 weeks
Prior stereotactic or gamma knife radiosurgery or proton radiation, unless unequivocal progression by functional neuro-imaging (PET, dynamic MRI, MRS, SPECT) or by re-operation with documented histologic confirmation of recurrence.
Prior anti-tumor therapy, as follows: at least 12-weeks from radiation therapy; at least 4-weeks from prior treatment with temozolomide or bevacizumab, 6-weeks from BCNU or CCNU.
Women of child bearing potential (WOCBP) who do not consent to using acceptable methods of birth control (oral contraceptives, IUD). For purposes of this study, WOCBP include any female who has experienced menarche, who has not undergone tubal ligation, and who is not postmenopausal. Post menopause is defined as: amenorrhea≥12 consecutive months without another cause.
Medically uncontrolled Type 1 or Type 2 diabetes mellitus
Pregnancy or lactation
Current participation in any other investigational clinical trial within 4-weeks.
Eastern Cooperative Oncology Group (ECOG) performance status>2 after optimization of medications (See Appendix 4) or KPS<60
Anticipated Life expectancy less than 3 months
Contraindications to the investigational product or known or suspected hypersensitivity
Patients who must take concomitant medications which induce or are potent inhibitors of CYP2C9 or sensitive substrates of CYP3A4 with narrow therapeutic range may not participate
Lack of suitability for participation in the trial, for any reason, as judged by the Investigator

Study Design:

The trial will be divided in two phases. In the first phase, 10-20 patients will be enrolled and treated with 300-520 mg BID of a compound of Formula (I) or (II) and VE821 for 28 days. The primary endpoint of the first phase is to determine the recommended Phase 2 dose (RP2D) of the compound of Formula (I) or (II)/VE821 combination in patients with recurrent or progressive glioblastoma and to assess the safety and toxicity of the compound of Formula (I) or (II)/VE821 combination in this patient population. The study has a 3+3 design and the first cohort will be treated with a compound of Formula (I) or (II) and VE821 for 28 days repeated in up to 5 cycles. If dose-limiting toxicity (DLT) such as neutropenia occurs, dosing will be interrupted and the individual patient will, following normalization, be restarted on the same or a lower dose level according to standardized procedure. If two or three of the first 3 patients on a specific dose level experience a DLT during the first 28 days of treatment with the compound of Formula (I) or (II), the following patients will be treated with a lower dose level. If one DLT occurs during the first 28 days of dosing in the first 3 three patients another 3 patients will be treated with the same dose level. If 2 of the 6 patients display DLT, the next patients will be treated with a lower dose level. The highest dose level without DLT or with maximally one DLT out of 6 patients will be the RP2D. All assessments with respect to dose adjustments for subsequent cohorts will be done during the first 28 days of treatment. Non-progressing patients may be treated for a total of five 28-day cycles (24 weeks).

In the second phase, 12 patients will be enrolled and treated with the identified RP2D of the compound of Formula (I) or (II)/VE821 combination for 28 days repeated in five cycles. The primary endpoints of phase II is to assess the proportion of patients who are progression-free at 24 weeks and to assess safety, tolerability, and adverse event profile of the compound of Formula (I) or (II)/VE821 combination.

Primary Outcomes:
Phase I—Determine recommended Phase II dose. [Time Frame: 8 months] [Designated as safety issue: Yes]
Phase II—Determine Antitumor effect [Time Frame: 4 months] [Designated as safety issue: Yes]
Phase I—Number of Participants with Adverse Events as a Measure of Safety and Tolerability [Time Frame: 6 months] [Designated as safety issue: Yes]
   physical/neurological examinations (pathological findings and quality and quantity)
   adverse events (quality and quantity per dose level)
   vital signs, ECG, laboratory parameters (pathological findings as quality and quantity, for laboratory parameters, descriptive statistics)

Secondary Outcomes:
Renal Phase I—Maximum Tolerated Dose (MTD) [Time Frame: 8 months] [Designated as safety issue: Yes]
To identify the MTD of a compound of Formula (I) or (II).
Phase I—Molecular markers of optimum response [Time Frame: 8 months] [Designated as safety issue: Yes]
To assess potential molecular markers that might predict optimum response sub-population groups
Phase I—Molecular Markers of IGF (insulin like growth factor)-1R pathway [Time Frame: 8 months] [Designated as safety issue: Yes]
To evaluate surrogate molecular markers of IGF-1R pathway activation/inhibition after treatment with compound of Formula (I) or (II)/VE821 combination in patients with malignant astrocytomas
Phase II—Time-To-Progression (TTP) and Overall Survival (OS) [Time Frame: 4 months] [Designated as safety issue: Yes]
To determine time-to-progression (TTP) and overall survival (OS) of patients treated with compound of Formula (I) or (II)/VE821 combination
Phase II—Overall Response Rate [Time Frame: 4 months] [Designated as safety issue: Yes]
To assess overall response rate (ORR) in recurrent malignant astrocytomas after treatment with the compound of Formula (I) or (II)/VE821 combination
Phase II—Imaging Evidence of Response. [Time Frame: 4 months] [Designated as safety issue: Yes]
To identify surrogate imaging evidence of response on MRI (magnetic resonance imaging)sequences by RANO criteria (with additional special attention to T2-FLAIR, DWI (diffusion-weighted imaging), perfusion MRI and multi-voxel MRS (magnetic resonance spectroscopy) sequences).

The examples and embodiments described herein are for illustrative purposes only and in some embodiments, various modifications or changes are to be included within the purview of disclosure and scope of the appended claims.

What is claimed is:

1. A pharmaceutical composition comprising 1) a first therapeutic agent, 2) a second therapeutic agent, and 3) at least one pharmaceutically acceptable excipient, wherein the first therapeutic agent is a compound of Formula (I), or a pharmaceutically acceptable salt, solvate, or prodrug thereof, having the structure: 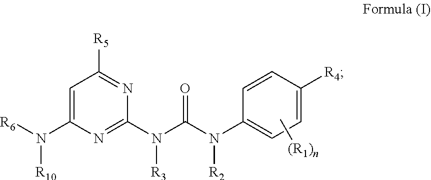
wherein:
each R₁ is independently halogen, —CN, —NO₂, —OH, —OCF₃, —OCH₂F, —OCF₂H, —SR₈, —N(R₈)S(═O)₂R₉, —S(═O)₂N(R₈)₂, —S(═O)R₉, —S(═O)₂R₉, —C(═O)R₉, —CO₂R₈, —N(R₈)₂, —C(═O)N(R₈)₂, —N(R₈)C(═O)R₉, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl;
or two R₁ are taken together to form a substituted or unsubstituted heterocyclic ring or a substituted or unsubstituted carbocyclic ring;
R₂ and R₃ are each independently H, —CN, C₁-C₄alkyl, C₃-C₆cycloalkyl, or C₂-C₇heterocycloalkyl; or R₂ and R₃ are taken together to form a 5- or 6-membered heterocyclic ring;
R₄ is H, halogen, —CN, —NO₂, —OH, —OCF₃, —OCH₂F, —OCF₂H, —SR₈, —N(R₈)S(═O)₂R₉, —S(═O)₂N(R₈)₂, —S(═O)R₉, —S(═O)₂R₉, —C(═O)R₉, —CO₂R₈, —N(R₈)₂, —C(═O)N(R₈)₂, —N(R₈)C(═O)R₉, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl;
R₅ is halogen, —CN, —OH, —CF₃, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl;
R₆ is —(C(R₁₄)(R₁₅))ₘN(R₁₁)(R₁₂);
R₁₁ and R₁₂ are each independently H, or substituted or unsubstituted C₁-C₆alkyl; or R₁₁ and R₁₂ are taken together to form a substituted or unsubstituted 5-, 6-, 7-, or 8-membered heterocyclic ring;
each R₁₄ and R₁₅ are each independently H, or substituted or unsubstituted C₁-C₆alkyl; or
R₁₄ and R₁₅ are taken together to form a 4-, 5-, 6-membered cycloalkyl ring;
each R₈ is independently H, or substituted or unsubstituted C₁-C₆alkyl;
each R₉ is independently substituted or unsubstituted C₁-C₆alkyl;
R₁₀ is H, or C₁-C₄alkyl;
m is 2-6; and
n is 0-4.

2. The pharmaceutical composition of claim 1, wherein R₂ and R₃ are each independently H, —CN, C₁-C₄alkyl, C₃-C₆cycloalkyl, or C₂-C₇heterocycloalkyl.

3. The pharmaceutical composition of claim 2, wherein R₂ and R₃ are each H.

4. The pharmaceutical composition of claim 1, wherein R₂ and R₃ are taken together to form a 5- or 6-membered heterocyclic ring.

5. The pharmaceutical composition of claim 4, wherein R₂ and R₃ are taken together to form a 5-membered heterocyclic ring.

6. The pharmaceutical composition of any one of claims 1-5, wherein R₆ is —(C(R₁₄)(R₁₅))ₘN(R₁₁)(R₁₂) and R₁₄ and R₁₅ are each H.

7. The pharmaceutical composition of claim 6, wherein R₁₁ and R₁₂ are each independently H, or substituted or unsubstituted C₁-C₆alkyl.

8. The pharmaceutical composition of claim 7, wherein R₁₁ and R₁₂ are each independently unsubstituted C₁-C₆alkyl.

9. The pharmaceutical composition of claim 8, wherein R₁₁ and R₁₂ are each —CH₃.

10. The pharmaceutical composition of any one of claims 1-9, wherein m is 2.

11. The pharmaceutical composition of any one of claims 1-9, wherein m is 3.

12. The pharmaceutical composition of any one of claims 1-11, wherein R₁₀ is H or CH₃.

13. The pharmaceutical composition of any one of claims 1-12, wherein R₈ is substituted or unsubstituted C₁-C₆alkyl.

14. The pharmaceutical composition of claim 13, wherein R₅ is CH₃.

15. The pharmaceutical composition of claim 13, wherein R₈ is CH₂CH₃.

16. The pharmaceutical composition of any one of claims 1-15, wherein R₄ is H, halogen, —CN, —NO₂, —OH, —OCF₃, —OCH₂F, —OCF₂H, —SR₈, —N(R₈)S(═O)₂R₉, —S(═O)₂N(R₈)₂, —S(═O)R₉, —S(═O)₂R₉, —C(═O)R₉, —CO₂RR, —N(R₈)₂, —C(═O)N(R₈)₂, —N(R₈)C(═O)R₉, substituted or unsubstituted C₁-C₆alkyl, or substituted or unsubstituted C₁-C₆alkoxy.

17. The pharmaceutical composition of claim 16, wherein R₄ is H, halogen, —CN, —OH, —OCF₃, substituted or unsubstituted C₁-C₆alkyl, or substituted or unsubstituted C₁-C₆alkoxy.

18. The pharmaceutical composition of claim 16, wherein R₄ is H, halogen, —OCF₃, substituted or unsubstituted C₁-C₆alkyl, substituted or unsubstituted C₁-C₆alkoxy.

19. The pharmaceutical composition of claim 16, wherein R₄ is halogen.

20. The pharmaceutical composition of any one of claims 1-19, wherein each R₁ is independently halogen, —CN, —NO₂, —OH, —OCF₃, —OCH₂F, —OCF₂H, —SR₈, —N(R₈)S(═O)₂R₉, —S(═O)₂N(R₈)₂, —S(═O)R₉, —S(═O)₂R₉, —C(═O)R₉, —CO₂R₈, —N(R₈)₂, —C(═O)N(R₈)₂, —N(R₈)C(═O)R₉, substituted or unsubstituted C₁-C₆alkyl, or substituted or unsubstituted C₁-C₆alkoxy.

21. The pharmaceutical composition of claim 20, wherein each R₁ is independently halogen, —CN, —OH, —OCF₃, substituted or unsubstituted C₁-C₆alkyl, or substituted or unsubstituted C₁-C₆alkoxy.

22. The pharmaceutical composition of claim 20, wherein each R₁ is independently halogen, —OCF₃, substituted or unsubstituted C₁-C₆alkyl, substituted or unsubstituted C₁-C₆alkoxy.

23. The pharmaceutical composition of claim 20, wherein each R₁ is independently is halogen.

24. The pharmaceutical composition of any one of claims 1-23, wherein n is 1.

25. The pharmaceutical composition of any one of claims 1-19, wherein n is 0.

26. The pharmaceutical composition of any one of claims 1-15, wherein n is 0 and R₄ is H, —CN, —NO₂, —OH, —OCF₃, —OCH₂F, —OCF₂H, —SR₈, —N(R₈)S(═O)₂R₉, —S(═O)₂N(R₈)₂, —S(═O)R₉, —S(═O)₂R₉, —C(═O)R₉, —CO₂R₈, —N(R₈)₂, —C(═O)N(R₈)₂, —N(R₈)C(═O)R₉, substituted or unsubstituted C₁-C₆alkyl, substituted or unsubstituted C₁-C₆alkoxy, substituted or unsubstituted C₁-C₆heteroalkyl, substituted or unsubstituted C₂-C₇heterocycloalkyl, substituted or unsubstituted C₃-C₈cycloalkyl, substituted or unsubstituted C₆-C₁₀aryl, or substituted or unsubstituted C₂-C₇heteroaryl.

27. The pharmaceutical composition of claim 26, wherein R₄ is H, —CN, —OH, —OCF₃, substituted or unsubstituted C₁-C₆alkyl, or substituted or unsubstituted C₁-C₆alkoxy.

28. The pharmaceutical composition of claim 1, wherein the compound of Formula (I) has the structure: 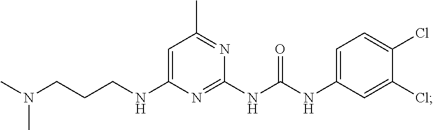 [SMILES: Cc1cc(NCCCN(C)C)nc(NC(=O)Nc2ccc(Cl)c(Cl)c2)n1]
or a pharmaceutically acceptable salt, solvate, or prodrug thereof.

29. A pharmaceutical composition comprising 1) a first therapeutic agent, 2) a second therapeutic agent, and 3) at least one pharmaceutically acceptable excipient, wherein the first therapeutic agent has the structure: 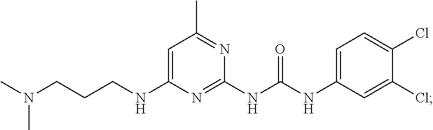 [SMILES: Cc1cc(NCCCN(C)C)nc(NC(=O)Nc2ccc(Cl)c(Cl)c2)n1]
or a pharmaceutically acceptable salt, solvate, or prodrug thereof.

30. The pharmaceutical composition of any one of claims 1-29, wherein the second therapeutic agent is an epidermal growth factor receptor (EGFR) inhibitor, an ataxia telangiectasia mutated (ATM) kinase inhibitor, an ataxia telengiectasia and Rad3 related (ATR) kinase inhibitor, a poly ADP-ribose polymerase (PARP) inhibitor, a cyclin-dependent kinase (CDK) 4/6 inhibitor, a checkpoint kinase 1 (Chk1) inhibitor, a signal transducer and activator of transcription 3 (STAT3) inhibitor, a mechanistic target of rapamycin (mTOR) inhibitor, or a Janus Kinase 2 (JAK2) inhibitor.

31. The pharmaceutical composition of any one of claims 1-30, wherein the second therapeutic agent is an epidermal growth factor receptor (EGFR) inhibitor.

32. The pharmaceutical composition of any one of claims 1-30, wherein the second therapeutic agent is an ataxia telangiectasia mutated (ATM) kinase inhibitor.

33. The pharmaceutical composition of any one of claims 1-30, wherein the second therapeutic agent is an ataxia telengiectasia and Rad3 related (ATR) kinase inhibitor.

34. The pharmaceutical composition of any one of claims 1-30, wherein the second therapeutic agent is a poly ADP-ribose polymerase (PARP) inhibitor.

35. The pharmaceutical composition of any one of claims 1-30, wherein the second therapeutic agent is a cyclin-dependent kinase (CDK) 4/6 inhibitor.

36. The pharmaceutical composition of any one of claims 1-30, wherein the second therapeutic agent is a checkpoint kinase 1 (Chk1) inhibitor.

37. The pharmaceutical composition of any one of claims 1-30, wherein the second therapeutic agent is a signal transducer and activator of transcription 3 (STAT3) inhibitor.

38. The pharmaceutical composition of any one of claims 1-30, wherein the second therapeutic agent is a mechanistic target of rapamycin (mTOR) inhibitor.

39. The pharmaceutical composition of any one of claims 1-30, wherein the second therapeutic agent is a Janus Kinase 2 (JAK2) inhibitor.

40. A method for treating cancer or Down's Syndrome in a subject comprising administering to the subject in need thereof a pharmaceutical composition of any one of claims 1-39.

41. The method of claim 40, wherein the disease is cancer.

42. The method of claim 41, wherein the cancer is brain cancer, glioblastoma multiforme, medulloblastoma, astrocytomas, brain stem gliomas, meningiomas, oligodendrogliomas, melanoma, lung cancer, breast cancer, or leukemia.

43. The method of claim 40, wherein the disease is Down's Syndrome.
